(19) United States
(12) Patent Application Publication
(10) Pub. No.: US 2018/0179166 A1
(43) Pub. Date: Jun. 28, 2018

(54) SMALL MOLECULE INHIBITORS OF GP120-MEDIATED HIV INFECTION AND METHODS OF USE

(71) Applicant: ViroCarb Inc., Toronto (CA)

(72) Inventors: Clifford LINGWOOD, Toronto (CA); Mario HUESCA, Thornhill (CA); Raveendra DAYAM, Hyderabad (IN); Shamshair SINGH, Hyderabad (IN)

(21) Appl. No.: 15/128,672

(22) PCT Filed: Mar. 24, 2015
(86) PCT No.: PCT/CA2015/000171

§ 371 Date: Sep. 23, 2016

Related U.S. Application Data
(60) Provisional application No. 61/971,404, filed on Mar. 27, 2014.

(51) Int. Cl. C07D 239/46; C07D 239/47; A61P 31/18; G01N 33/68; C07D 253/075; C07D 239/60; C07D 401/12; C07D 403/12; C07D 405/12; C07D 401/14; C07D 417/14; C07D 333/48; C07D 231/06; C07D 401/04

(52) U.S. Cl. CPC C07D 239/46; G01N 2500/02; A61P 31/18; G01N 33/6893; C07D 253/075; C07D 239/60; C07D 401/12; C07D 403/12; C07D 405/12; C07D 401/14; C07D 417/14; C07D 333/48; C07D 231/06; C07D 401/04; G01N 2333/162; C07D 239/47

(57) ABSTRACT

Small molecule inhibitors GP120-mediated HIV infections, methods for identifying the same, and methods and uses employing of these compounds in treatment, inhibition or prevention of HIV infection are provided. The compounds have a structure according to Formula (I), and comprise a pharmacophore functionally corresponding with pharmacophore (I) of FIG. 2.

### CROSS REFERENCE TO RELATED APPLICATIONS

This application claims the benefit of priority of U.S. Provisional Patent Application No. 61/971,404 filed Mar. 27, 2014, which is hereby incorporated by reference.

### FIELD

Technologies described herein pertain to the field of HIV treatment and prevention and in particular, to small molecule inhibitors of GP120-mediated HIV infections, methods for identifying the same, manufacture thereof and use of these compounds in treatment or prevention of HIV infection.

### BACKGROUND

HIV infections remain one of the most devastating pandemics of modern times. In the absence of a vaccine, intervention is restricted to drugs which selectively interfere with specific stages within the infectious life cycle of this retrovirus. These targets include viral RNA reverse transcription, integration into the host genome via inhibition of the viral integrase enzyme, protease inhibition to block maturation of viral proteins from a common larger precursor and inhibitors which block viral-host cell targeting and membrane fusion. Selection of drug resistant strains due to the high mutation rate of the HIV virus, eventually subverts all these drug interventions. Entry inhibitors are a relatively new attractive target since host cell factors, not susceptible to mutation, can be targeted.

Two entry inhibitors have been approved for clinical use. Maraviroc is a small molecule chemical agonist of the CCR5 chemokine receptor bound by R5 HIV-1 (Parra et al., 2011) and therefore it only targets one of the two subtypes of HIV strains, i.e., those that bind CCR5. Enfuvirtide is a peptide analogue of the heptad repeat fusion peptide of gp41 which blocks HIV host cell fusion (Joly et al., 2010). However enfuvirtide must be administered as an injectable. The soluble adamantylGb₃ analogue is effective to prevent infection by both X4 and R5 HIV-1 strains and drug resistant HIV strains retain sensitivity to this inhibition (Lund et al., 2006). In addition, Gb₃ has previously been identified as a natural host resistance factor against HIV infection in vitro (Lund et al., 2009). Gb₃-deficient lymphocytes are hypersensitive to HIV infection in vitro, while lymphocytes in which Gb₃ accumulates are relatively resistant in vitro to X4 and R5 HIV-1 infection (Lund et al., 2005; Lund et al., 2009).

The HIV virus binds CD4 positive human T cells due to the CD4 binding site on the HIV envelope glycoprotein gp120, which mediates virus-host cell binding. CD4 binding induces a conformational change in gp120 such that a binding site for the co-receptor (either the chemokine receptor CCR5, recognized by R5 HIV-1, or CXCR4, recognized by X4 HIV-1) is exposed within the V3 loop of gp120. This secondary co-receptor binding by the V3 loop of gp120 initiates host cell signal transduction pathways necessary for infection. Chemokine co-receptor binding induces a further conformational change within gp120 to expose the fusion peptide of the gp120 associated gp41, and thereby induce fusion of the viral and host plasma membrane and allow the virus to enter the cell.

The V3 loop of gp120 also encodes a prototypic glycosphingolipid binding site (Delezay et al., 1996; Mahfoud et al. 2002) which mediates binding of gp120 to several glycosphingolipids (GSLs), most notably globotriaosylceramide (Gb₃), monosialoganglioside(GM3), sulfogalactosyl ceramide(SGC) and galactosyl ceramide(GalCer) which can be expressed in the host cell membranes. GSLs are primarily found within lipid rafts (in which GSLs and cholesterol accumulate) and such rafts are required for HIV infection (Liao et al., 2001). This interaction between gp120 and GSLs has been generally thought to promote the simultaneous binding of CD4 and chemokine receptor by gp120. In the absence of CD4, GSL binding has been shown to mediate the less effective infection of CD4 negative cells by HIV (Fantini et al., 1993).

FIG. 1 illustrates the known V3 loop of gp120. Amino acids within the V3 loop GSL binding site are emphasized adjacent the letters “G, P, G” and “R, A, F”, originally designated by colors yellow and green, respectively (color not shown). Amino acids required for CCR5 binding are emphasized adjacent the letters “R, A, F” and “S, E”, originally designated by colors green and red (colors not shown). The Uniprot ID of GP120 (V3 loop) is P35961.

Amino acids within the GSL binding site in the V3 loop are also required for chemokine receptor binding and it has been previously proposed this gp120-GSL interaction is in fact, an inhibitory event, reducing the ability of the V3 loop to bind to chemokine co-receptor. Soluble analogues of Gb₃, GM3 and GalCer have been synthesized which show inhibition of HIV infection and host cell fusion.

Structural studies on gp120 indicate that chemokine receptor N terminus binds primarily to the base of the V3 loop but also that the disorder within the tip and stem of the V3 loop become more ordered after co-receptor binding (Huang et al., 2007). Extracellular loop 2 of the chemokine receptor binds the V3 loop tip (Huang et al., 2007). The tip of the V3 loop contains the GSL binding site (as shown in FIG. 1) (Mahfoud et al., 2002). Both the tip and base sequences of the V3 loop have been proposed as overlooked conserved targets for HIV therapeutic development (Andrianov, et al. 2011).

Interaction of HIV-1 gp120 envelope glycoprotein with the primary receptor, CD4, promotes binding to a chemokine receptor, either CCR5 or CXCR4. The chemokine receptor-binding site on gp120 elicits CD4-induced (CD4i) antibodies in some HIV-1-infected individuals. The antibody 412d is one such antibody. It binds to a CD4-induced epitope that overlaps the site of co-receptor binding on HIV-1 gp120, and recognizes preferentially CCR5-dependent strains of HIV-1 gp120. The CD4i antibody 412d utilizes sulfated tyrosines to achieve binding to gp120.

This background information is provided for the purpose of making known information believed by the applicant to be of possible relevance. No admission is necessarily intended, nor should be construed, that any of the preceding information constitutes prior art.

It is desirable to provide a molecule for preventing, inhibiting or treating GP120-mediated HIV infection.

### SUMMARY

Compounds capable of preventing, inhibiting or treating HIV infection are described herein, having a pharmacophore functionally corresponding to the interaction of HIV-1 gp120 with portion of an exemplary antibody 412d. A pharmacophore model is provided for identifying such compounds. Compositions and methods pertaining to the compounds, including the use and identification thereof, are provided.

In accordance with one aspect, there is provided a compound of formula (I)

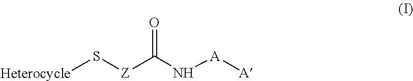

or a pharmaceutically acceptable salt thereof, for use in treating, inhibiting, or preventing HIV infection in a subject, wherein:

A is absent or is —N═CH—;

Z is —CH₂— or —CH₂—CH₂—;

A′ is C₆-C₁₀ aryl or heteroaryl, optionally substituted with one or more of C₁-C₆ alkyl, —OH, —O—C₁-C₆ alkyl, —OCX₃, halogen, —C(O)OR, —SO₂NR′R″, —NR′R″ or —O—(CH₂)₁₋₃—O— to form a bicyclic ring; wherein X is halogen, R is C₁-C₆ alkyl, R′ and R″ are independently H, C₁-C₆ alkyl, or R′ and R″ taken together with N form a C₃-C₇ heterocycle comprising one or more additional hetero atoms selected from N and O; and

heterocycle(s) are substituted or unsubstituted;

wherein the compound comprises a pharmacophore functionally corresponding with pharmacophore I of FIG. 2.

Further, there is provided herein a compound or a pharmaceutically acceptable salt thereof, for use in treating, inhibiting, or preventing HIV infection in a subject, wherein the compound is one according to any one of compounds #1 to #56.

Additionally, there is provided herein a method of treating, inhibiting, or preventing HIV infection comprising administering a therapeutically effective amount of a compound of formula (I):

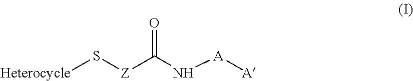

or a pharmaceutically acceptable salt thereof to a subject, wherein Z, A, A′, and the heterocycle are as described above, wherein the compound comprises a pharmacophore functionally corresponding with pharmacophore I of FIG. 2.

There is provided a method of treating, inhibiting, or preventing HIV infection comprising administering to a subject an effective amount of any of compounds #1 to #56.

Additionally, there is provided a method of identifying a compound for binding with the glycosphingolipids (GSL) binding site at the V3-loop tip of gp120 of HIV, and for inducing the conformational change following chemokine receptor binding to inhibit chemokine receptor binding, said method comprising: screening a small molecule library using in silico high throughput docking for candidate compounds having a conformation capable of targeting and disrupting co-receptor binding or having a pharmacophore functionally corresponding with pharmacophore I of FIG. 2; and evaluating the candidate compounds for the ability to inhibit HIV infection to identify a compound for binding with the GSL binding site.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 presents the V3 loop of gp120. Amino acids within the V3 loop GSL binding site are emphasized adjacent the letters “G, P, G” and “R, A, F”.

FIG. 2 illustrates the key features of pharmacophore I, showing key interactions between gp120 and the 412d antibody.

FIG. 3 illustrates AdamantylGSLs inhibition of gp120 binding to CD4/CCR5 transfected U87 cells.

FIG. 4 illustrates a mapping of variations in the V3 loop sequence.

FIG. 5 illustrates a mapping of the V3 loop sequence, in particular FARGPG at positions 312-317.

FIG. 6 illustrates crystal structure of CD4 induced gp120 complex with 412d

FIG. 7 illustrates a conformational change in gp120 induced by CCR5 binding and relation to GSL binding.

FIG. 8 illustrates the virtual screening protocol using a pharmacophore model based on HIV-1 gp120 interaction with antibody 412d.

FIG. 9 illustrates pharmacophore I interactions and residues, showing key interactions between gp120 and the 412d antibody, as depicted in FIG. 2.

FIG. 10 shows GP-120 V3-loop interactions of compound #52.

FIG. 11 shows GP-120 V3-loop interactions of compound #53.

FIG. 12 shows GP-120 V3-loop interactions of compound #54.

FIG. 13 shows GP-120 V3-loop interactions of compound #55.

FIG. 14 shows GP-120 V3-loop interactions of compound #56.

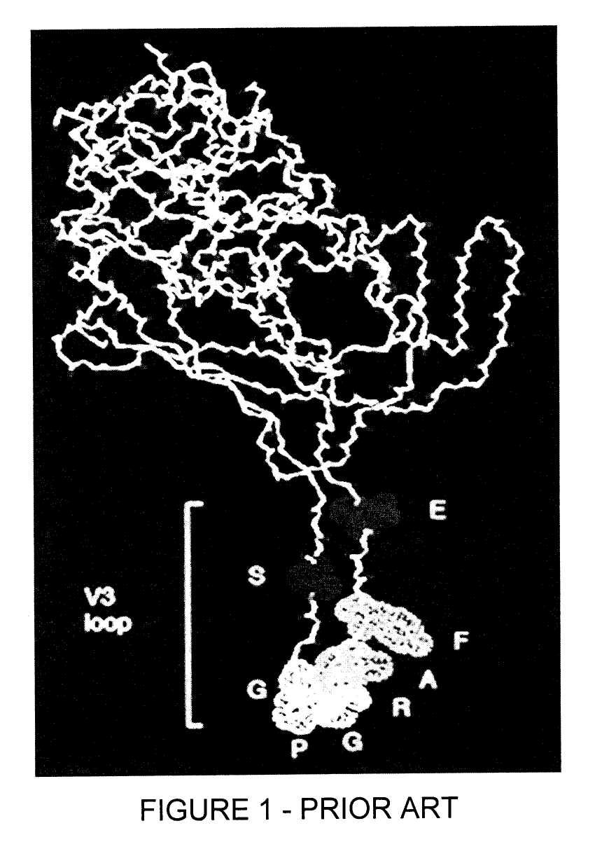

### DETAILED DESCRIPTION

There is described herein molecules, together with methods and uses therefor in preventing, inhibiting or treating GP120-mediated HIV infection.

Using a pharmacophore model, a number of molecules were identified from small molecules databases having an appropriate pharmacophore structure as candidate inhibitors of GP120-mediated HIV infection. Selected molecules were screened in biological assays.

Small-molecule inhibitors of GP120 interaction with co-receptors (CCR5 & CXCR4) are described. HIV-1 entry into host cell is a complex process. HIV-1 entry into host cells needs binding of its gp120 envelope glycoprotein to cell-surface receptors, CD4 and a co-receptor, either CCR5 or CXCR4. After binding to CD4, gp120 undergoes major conformational changes and exposes its V3 loop base for co-receptors. Posttranslational O-sulfation of N-terminal tyrosine residues of co-receptors is required for their interaction with HIV-1 gp120. Particularly, O-sulfation of N-terminal tyrosine residues 10 and 14 is sufficient for interaction of CCR5 with V3 loop base of gp120. The analysis of co-crystal structure of CD4 induced gp120 complex with antibody 412d bearing sulfated tyrosine residues revealed interaction of the O-sulfated tyrosines with V3 loop base of gp120. The observed interactions between O-sulfated tyrosines of 412d with V3 loop base of gp120 represent co-receptor interaction with gp120. Small molecules that bind at the base of the gp120 V3 loop with high affinity will disrupt gp120 interaction with co-receptors. Blocking of the base of the gp120 V3 loop with high affinity small molecules inhibits HIV-1 entry into host cell. In silico design strategies are provided to identify small molecule inhibitors of gp120 interaction with co-receptors, based on pharmacophore I.

A co-receptor interaction-based pharmacophore is provided herein. The CCR5 binding site on gp120 was considered for structure-based pharmacophore modeling. Interaction of the sulfotyrosines (Tyr-Arg-Asp-Tyr amino acids) bearing region of 412d with the base of the V3 loop was the template for gp120 interactions with co-receptor (PDB entry: 2QAD). The key interactions observed between gp120 and CCR5 were converted into 3D pharmacophore features. These features were reasonably combined to generate a pharmacophore model. The pharmacophore was used to retrieve novel compounds with diverse chemical scaffolds from databases of small-molecules, which were subjected to further testing.

There is provided a compound of formula (I)

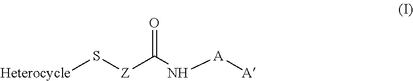

or a pharmaceutically acceptable salt thereof, for use in treating, inhibiting, or preventing HIV infection in a subject, wherein:

A is absent or is —N═CH—;

Z is —CH₂— or —CH₂—CH₂—;

A′ is C₆-C₁₀ aryl or C₆-C₁₀ heteroaryl, optionally substituted with one or more of C₁-C₆ alkyl, —OH, —O—C₁-C₆ alkyl, —OCX₃, halogen, —C(O)OR, —SO₂NR′R″, —NR′R″ or —O—(CH₂)₁₋₃—O— to form a bicyclic ring; wherein X is halogen, R is C₁-C₆ alkyl, R′ and R″ are independently H, C₁-C₆ alkyl, or R′ and R″ taken together with N form a C₃-C₇ heterocycle comprising one or more additional hetero atoms selected from N and O; and

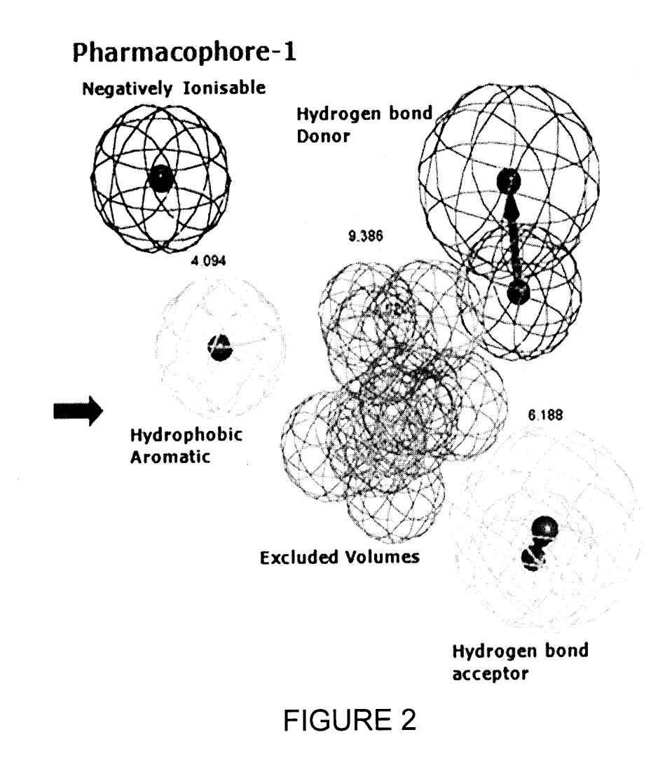

heterocycle(s) are substituted or unsubstituted;

wherein the compound comprises a pharmacophore functionally corresponding with pharmacophore I of FIG. 2.

To have a pharmacophore that functionally corresponds with pharmacophore-I, the pharmacophore of the compound permits some inhibitory binding to gp120. This functional correspondence may be attributed to the compound possessing at least one of the features of the pharmacophore-I model, specifically: a negatively ionizable feature, a hydrophobic aromatic feature, a hydrogen bond acceptor feature, and a hydrogen bond donor feature; and/or by having similar inter-feature distances. Such common features allow the compound to possess functional correspondence to the pharmacophore, and thereby possess similar functional features allowing HIV gp120 binding.

The above compound may have an aryl group that is phenyl or naphthyl, optionally substituted with one or more of said substituents. The aryl group of the compound (when present) may be phenyl, substituted at the p-position. Further, the aryl group may be phenyl, substituted with one or more substituent selected from the group consisting of: methyl, ethyl, propyl, isopropyl, butyl, —F, —Cl, —OH, —O-n-propyl, —O-isopropyl, —C(O)OH, —C(O)OCH₃, —C(O)OCH₂CH₃, —O—CF₃, —O—CCl₃, —SO₂NH₂, and —SO₂NR′R″ wherein R′ and R″ are H or taken together with N atom to form a five a six membered ring, wherein said 5— or 6-membered ring optionally having an oxygen atom in addition to the N atom.

The compound may have an aryl group that is phenyl, substituted with —O—CH₂—O— or —O—(CH₂)₂—O— to form:

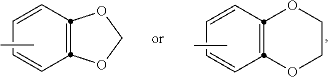

respectively.

For compounds in which A′ comprises a heteroaryl, the heteroaryl may be a pyridinyl group.

The heterocycle(s) encompassed within a compound of formula I (either at the far left of the molecule as represented above, or within the A′ moiety, may be substituted or unsubstituted. The heterocycle(s) may be a 5 or 6 membered heterocycle, comprising one or more ring nitrogen atoms, optionally substituted with one or more —OH, —NH₂ or oxo (C═O) groups, or other appropriate substituents independently at different positions on the ring.

The pharmacophore of the compounds described herein, is a portion of the compound having a biological or pharmacological interaction with the HIV virus, such as the V3 loop of HIV envelope glycoprotein gp 120. The pharmacophore described herein comprises a negatively ionizable feature, a hydrophobic aromatic feature, a hydrogen bond acceptor feature, and a hydrogen bond donor feature. Such features may possess an inter-feature distance of one or more of the following: (i) between the negatively ionizable feature and hydrophobic aromatic feature a distance of from about 3 to about 5 Å, or from about 3.5 to about 4.5 Å; (ii) between the hydrophobic aromatic feature and the hydrogen bond acceptor feature a distance of from about 7 to about 11 Å, or from about 8 to about 10 Å; (iii) between the negatively ionizable feature and hydrogen bond donor feature a distance of from about 7 to about 11 Å, or from about 8 to about 10 Å; or (iv) between the hydrogen bond donor feature and the acceptor feature a distance of from about 4 to about 8 Å, or from about 5 to about 7 Å.

For better understanding of the features of the compounds described herein, it should be understood that a negatively ionizsable (alternatively spelled: ionisable) feature may be the structural part of the molecule that is, or can be, negatively charged at physiological pH. For example, functional groups such as but not limited to a carboxylic acid is a tetrazole in structures may represent a “negatively ionisable” feature. A hydrophobic aromatic group encompasses structural portion of a molecule that include a hydrophobic portion that is also aromatic. Typical moieties include, but are not limited to phenyl, phenol, pyridine, pyrimidine, or pyrazine. A hydrogen bond acceptor is, generally, sp or sp² nitrogen that has a lone pair and a charge less than or equal to zero; a sp³ oxygen or sulfur that has a lone pair and a charge less than or equal to zero; or may be a non-basic amine that have a lone pair. The defining characteristic is the ability to accept a hydrogen bond. Exemplary structural moieties representing the hydrogen acceptor feature may include, but are not limited to pyrimidine, nitrogen, carbonyl oxygen of pyrimidi-4-one, nitrogen of 1,2,4 triazine, carbonyl oxygen of tetrahydrothiophene 1,1-dioxide, and nitrogen for example at the 3rd position in benzimidazole. An exemplary hydrogen bond donor as described herein may be a non-acidic hydroxyl, a thiol, an acetylenic hydrogen, or —NH, except —NH of tetrazoles and trifluoromethyl sulfonamide hydrogens. Sample hydrogen bond donor moieties are: the amino group of 6-amino-pyrimidinone, and the hydroxyl group of 1,2,4-triazine-3,5-diol.

The compounds described herein will typically possess each of the negatively ionizable, hydrogen bond acceptor, hydrogen bond donor and hydrophobic aromatic features. Should only three of the four features be present, two of the three should be the negatively ionizable and hydrogen acceptor features.

Excluded volumes within the compound may be present to ensure that other parts of the structure do not fall within (occupying) this region. The excluded volumes indicate that the structure of the compound does not extend into this region, according to the pharmacophore, so as to avoid unfavorable interactions between the structure and HIV-1 GP120 protein.

The compounds or pharmaceutically acceptable salts thereof described herein for use in treating, inhibiting, or preventing HIV infection in a subject, typically fall into the structure of Formula (I), but need not fall within Formula (I), provided the compound adheres to one of the following compounds (#1 to #56), or is a minor variant or functional derivative of one of these, and/or possesses pharmacophore-1, pharmacophore-1Å, or a functional equivalent thereto:

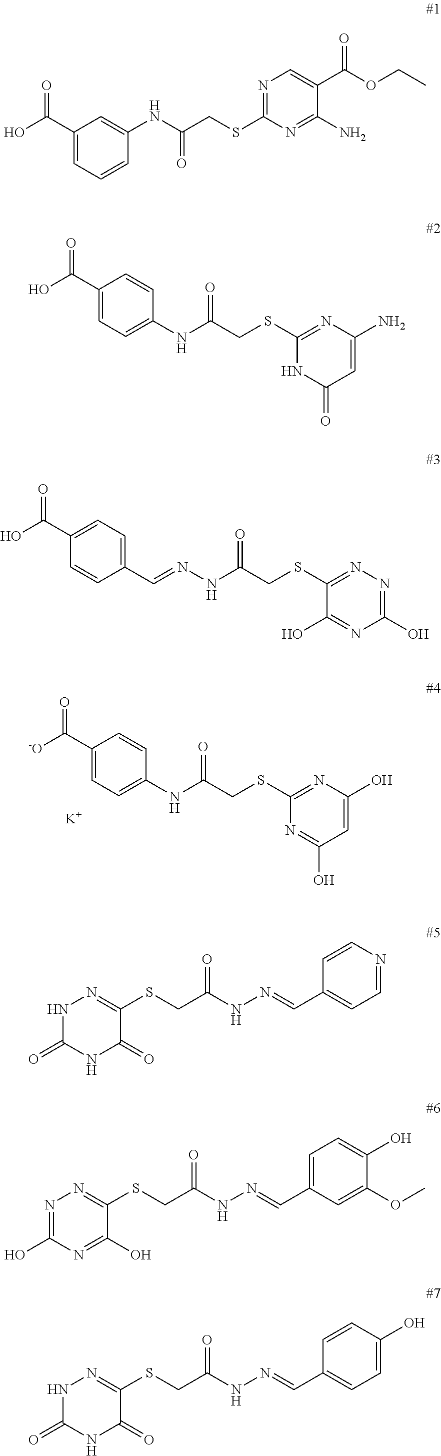

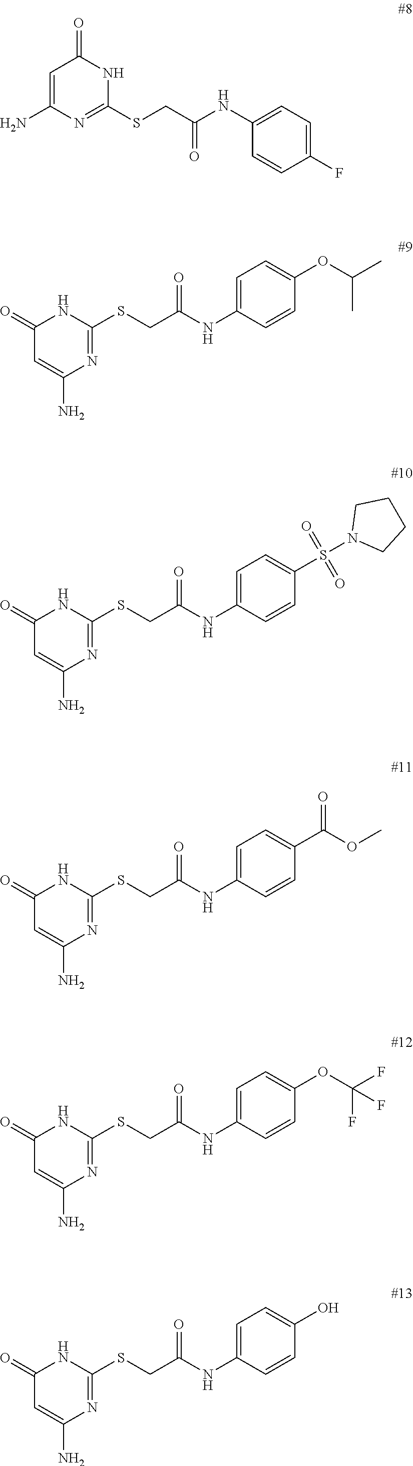 [SMILES: Nc1cc(=O)[nH]c(SCC(=O)Nc2ccc(F)cc2)n1 ; CC(C)Oc1ccc(NC(=O)CSc2nc(N)cc(=O)[nH]2)cc1 ; Nc1cc(=O)[nH]c(SCC(=O)Nc2ccc(S(=O)(=O)N3CCCC3)cc2)n1 ; COC(=O)c1ccc(NC(=O)CSc2nc(N)cc(=O)[nH]2)cc1 ; Nc1cc(=O)[nH]c(SCC(=O)Nc2ccc(OC(F)(F)F)cc2)n1 ; Nc1cc(=O)[nH]c(SCC(=O)Nc2ccc(O)cc2)n1]

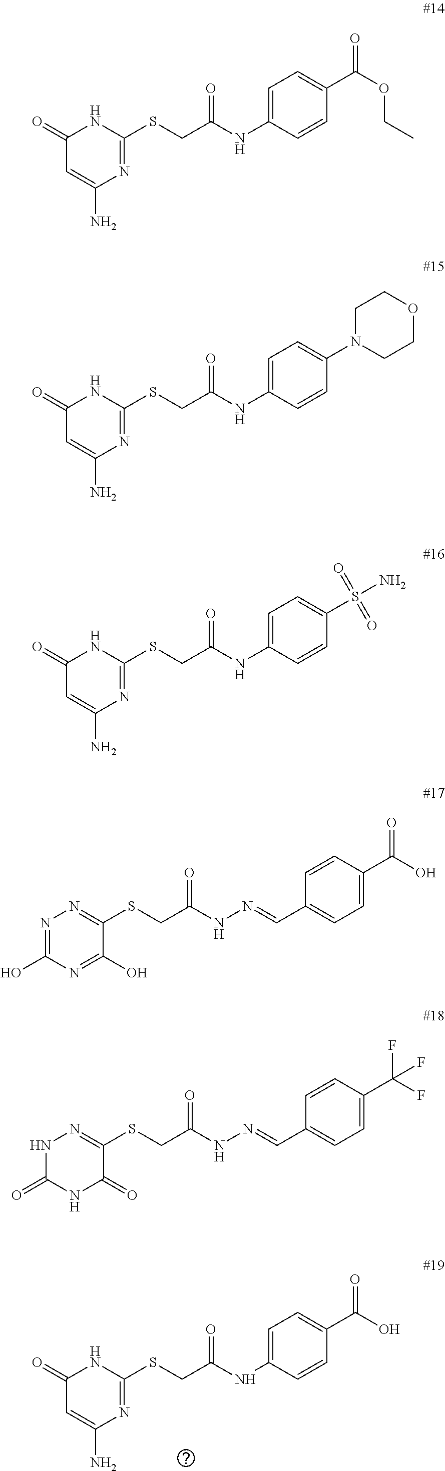 [SMILES: CCOC(=O)c1ccc(NC(=O)CSc2nc(N)cc(=O)[nH]2)cc1 ; Nc1cc(=O)[nH]c(SCC(=O)Nc2ccc(N3CCOCC3)cc2)n1 ; Nc1cc(=O)[nH]c(SCC(=O)Nc2ccc(S(N)(=O)=O)cc2)n1 ; O=C(CSc1nnc(O)nc1O)N/N=C/c1ccc(C(=O)O)cc1 ; O=C(CSc1n[nH]c(=O)[nH]c1=O)N/N=C/c1ccc(C(F)(F)F)cc1 ; Nc1cc(=O)[nH]c(SCC(=O)Nc2ccc(C(=O)O)cc2)n1]

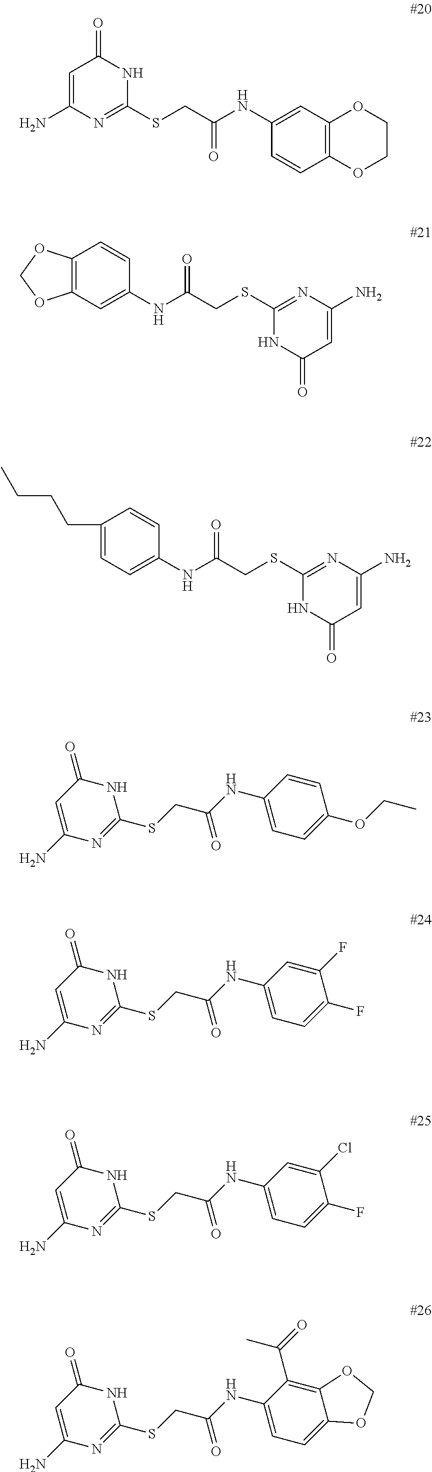

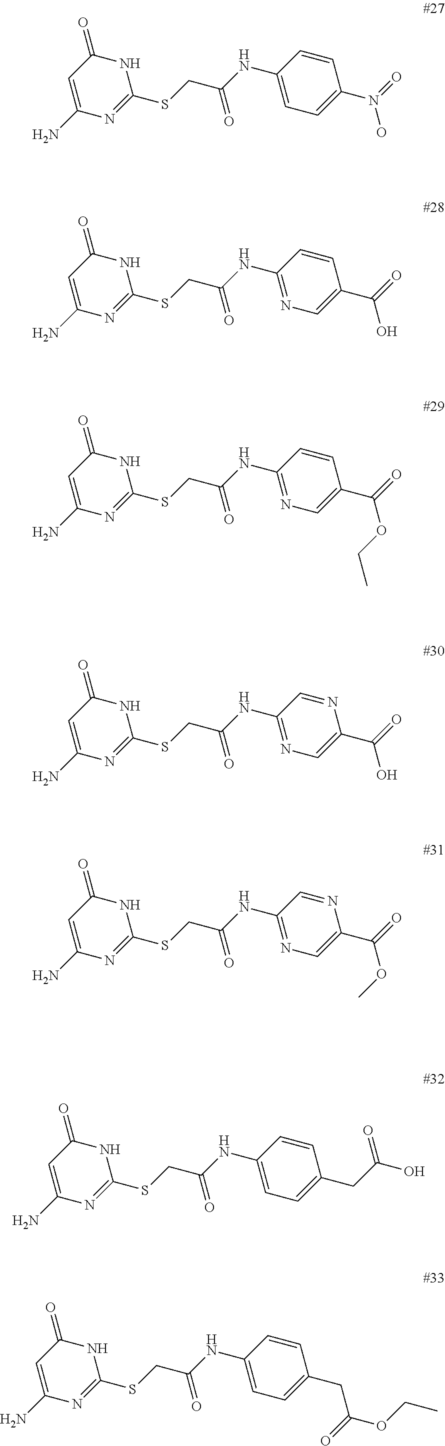

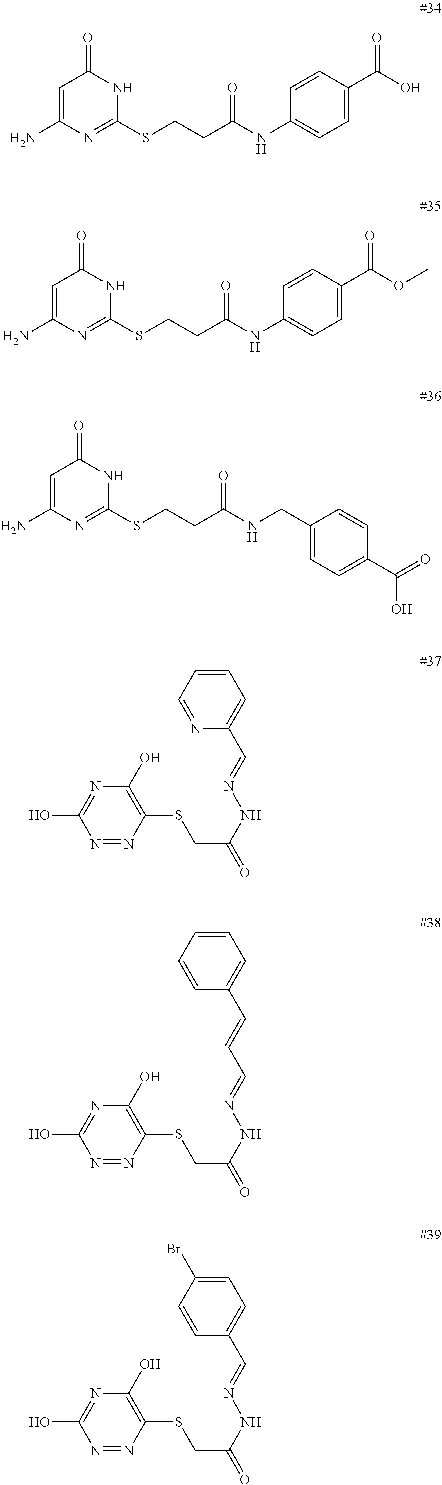 [SMILES: Nc1cc(=O)[nH]c(SCCC(=O)Nc2ccc(C(=O)O)cc2)n1 ; COC(=O)c1ccc(NC(=O)CCSc2nc(N)cc(=O)[nH]2)cc1 ; Nc1cc(=O)[nH]c(SCCC(=O)NCc2ccc(C(=O)O)cc2)n1 ; O=C(CSc1nnc(O)nc1O)N/N=C/c1ccccn1 ; O=C(CSc1nnc(O)nc1O)N/N=C/C=C/c1ccccc1 ; O=C(CSc1nnc(O)nc1O)N/N=C/c1ccc(Br)cc1]

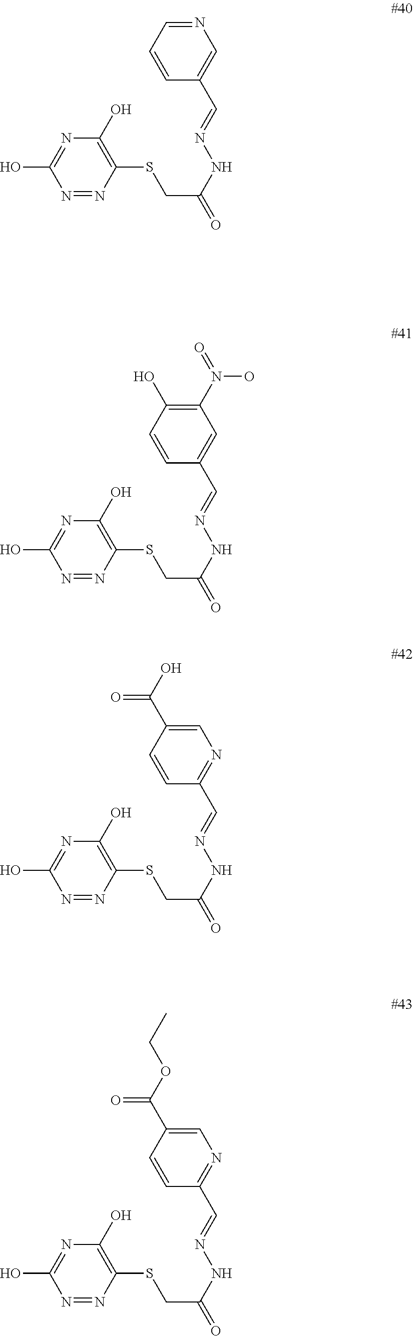

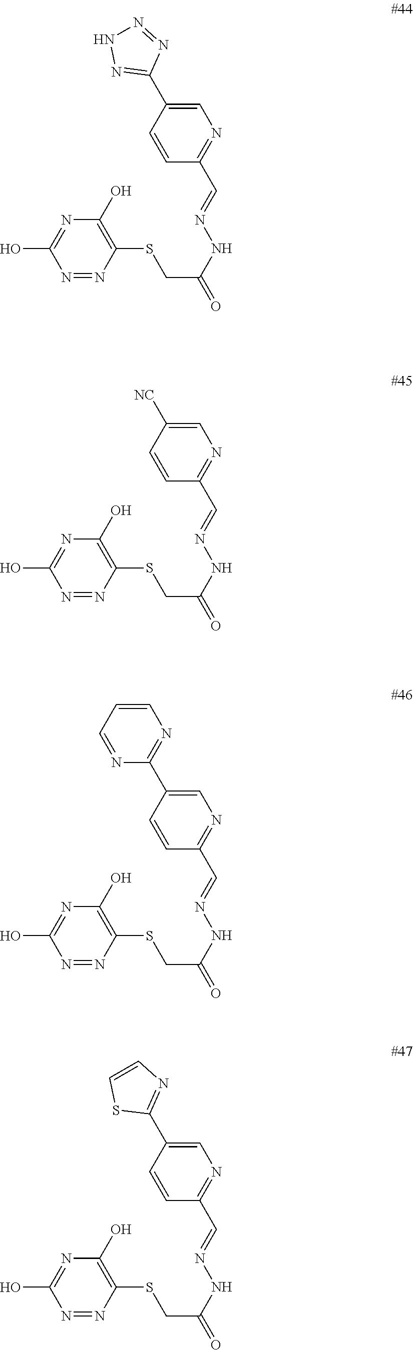 [SMILES: O=C(CSc1nnc(O)nc1O)N/N=C/c1ccc(-c2nn[nH]n2)cn1 ; N#Cc1ccc(/C=N/NC(=O)CSc2nnc(O)nc2O)nc1 ; O=C(CSc1nnc(O)nc1O)N/N=C/c1ccc(-c2ncccn2)cn1 ; O=C(CSc1nnc(O)nc1O)N/N=C/c1ccc(-c2nccs2)cn1]

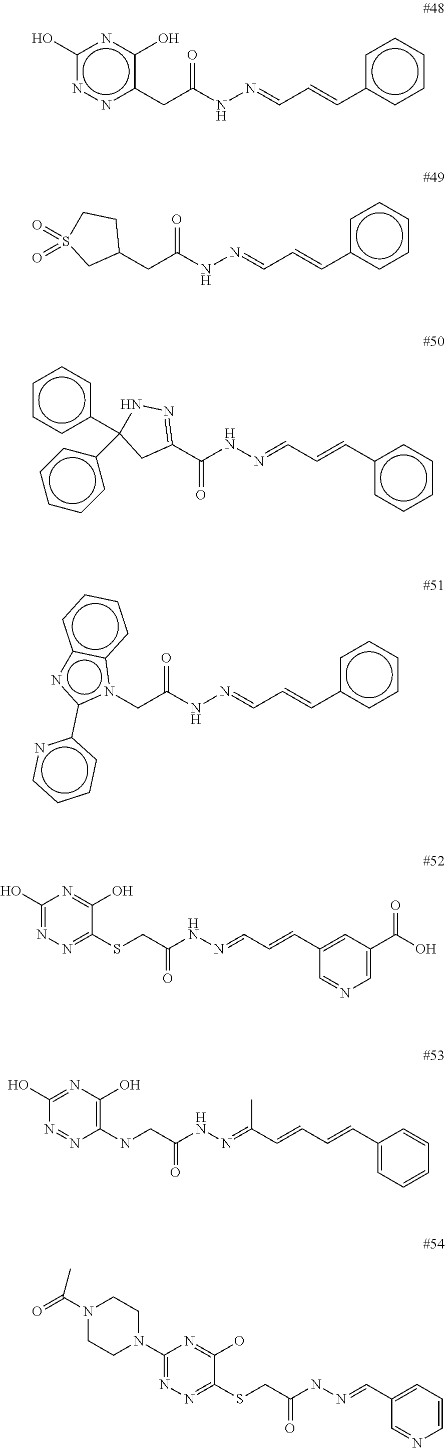

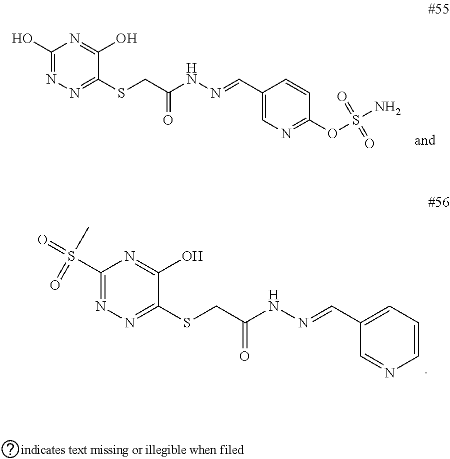 [SMILES: NS(=O)(=O)Oc1ccc(/C=N/NC(=O)CSc2nnc(O)nc2O)cn1 ; CS(=O)(=O)c1nnc(SCC(=O)N/N=C/c2cccnc2)c(O)n1]

A pharmaceutical composition is described, comprising any of compounds #1 to #56, or any of the compounds of formula (I), or salts thereof and a pharmaceutical carrier.

Method of treating, inhibiting, or preventing HIV infection are provided, comprising administering a therapeutically effective amount of a compound of formula (I) or a pharmaceutically acceptable salt thereof to a subject, provided the compound comprises a pharmacophore functionally corresponding with pharmacophore-I of FIG. 2.

A method of treating, inhibiting, or preventing HIV infection is described comprising administering to a subject an effective amount of one or more of compounds #1 to #56.

A method is provided for identifying compounds for binding to sequences within the V3-loop tip of gp120 of HIV that will induce conformational changes following chemokine receptor binding to inhibit chemokine receptor binding. The method comprises screening a small molecule library using in silico high throughput docking for candidate compounds having a conformation capable of targeting and disrupting co-receptor binding or having a pharmacophore functionally corresponding with pharmacophore I of FIG. 2; and evaluating the candidate compounds for the ability to inhibit HIV infection to identify a compound for binding with the GSL binding site.

In such a method, the small molecule library can particularly be screened for candidate compounds having a pharmacophore functionally corresponding with pharmacophore I of FIG. 2 in one or more aspect. For example, the pharmacophore comprises a negatively ionizable feature, a hydrophobic aromatic feature, a hydrogen bond acceptor feature, and a hydrogen bond donor feature. An exemplary pharmacophore may comprise an inter-feature distance of: (a) between the negatively ionizable feature and hydrophobic aromatic feature a distance of from about 3 to about 5 Å, or from about 3.5 to about 4.5 Å; (b) between the hydrophobic aromatic feature and the hydrogen bond acceptor feature a distance of from about 7 to about 11 Å, or from about 8 to about 10 Å; (c) between the negatively ionizable feature and hydrogen bond donor feature a distance of from about 7 to about 11 Å, or from about 8 to about 10 Å; and/or (d) between the hydrogen bond donor feature and the acceptor feature a distance of from about 4 to about 8 Å, or from about 5 to about 7 Å.

As used in this application, except as otherwise expressly provided herein, each of the following terms shall have the meaning set forth below.

“HIV” refers to the human immunodeficiency virus. HIV shall include, without limitation, HIV-1. HIV-1 includes but is not limited to extracellular virus particles and the forms of HIV-1 associated with HIV-1 infected cells. The human immunodeficiency virus (HIV) may be either of the two known types of HIV (HIV-1 or HIV-2). The HIV-1 virus may represent any of the known major subtypes (classes A, B, C, D, E, F, G, H, or J), outlying subtype (Group 0), or an as yet to be determined subtype of HIV-1. HIV-1_{JR-FL} is a strain that was originally isolated at autopsy from the brain tissue of an AIDS patient. The virus has been cloned and the DNA sequences of its envelope glycoproteins are known (GenBank Accession No. U63632).

“Administering” refers to delivering in a manner which is effected or performed using any of the various methods and delivery systems known to those skilled in the art. Administering can be performed, for example, topically, intravascularly, intravenously, pericardially, orally, parenterally, via implant, transmucosally, dermally, transdermally, intradermally, intramuscularly, subcutaneously, intraperitoneally, intrathecally, intralymphatically, intralesionally, epidurally, rectally, intravaginally, intraocularly, intrasinally, nasally, intraspinally, mucosally, transmucosally, transplacentally or by in vivo electroporation. An agent or composition may also be administered in an aerosol, such as for pulmonary and/or intranasal delivery. Administering can also be performed, for example, once, a plurality of times, and/or over one or more extended periods.

As used herein, “CCR5”, is a chemokine receptor which binds members of the C—C group of chemokines and whose amino acid sequence comprises that provided in Genbank Accession Number 1705896 and related polymorphic variants. As used herein, CCR5 includes, without limitation, extracellular portions of CCR5 capable of binding the HIV-1 envelope protein. “CCR5” and “CCR5 receptor” are used synonymously.

The term “pharmaceutically acceptable salt” as used herein, refers to a salt of a compound of Formula I, which is substantially non-toxic to living organisms. Typical pharmaceutically acceptable salts include those salts prepared by reaction of the compounds described herein with a pharmaceutically acceptable mineral or organic acid or an organic or inorganic base. Such salts are known as acid addition and base addition salts.

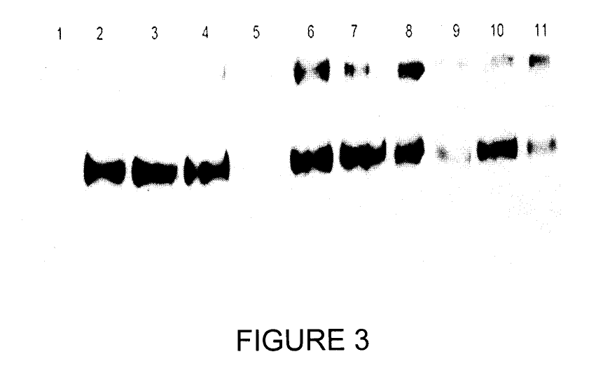

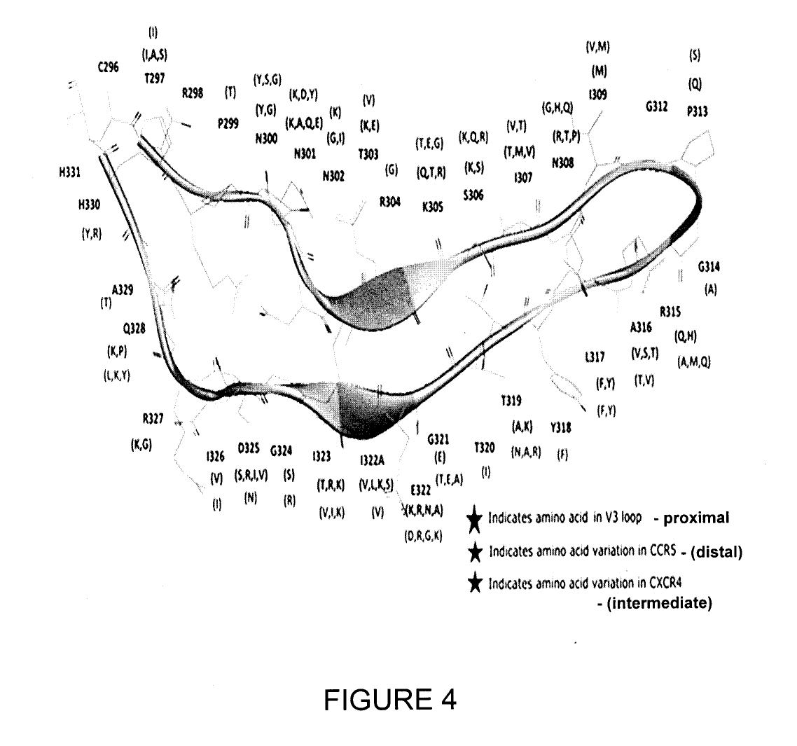

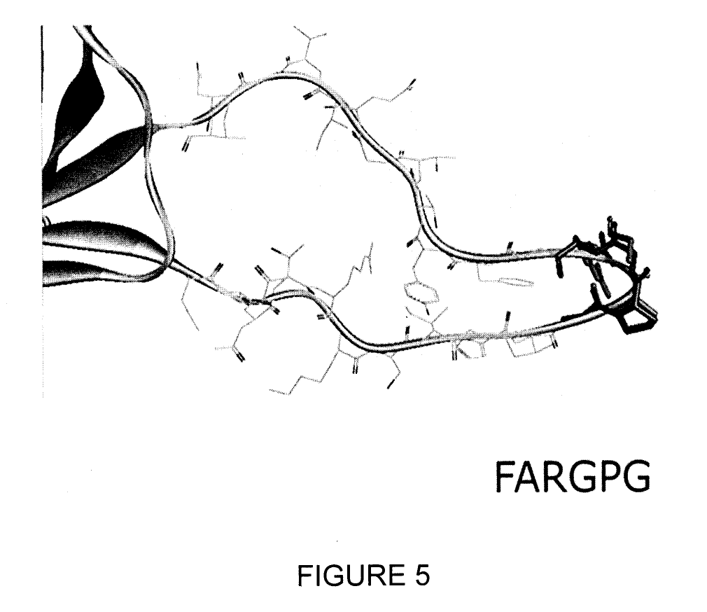

There is described herein an in silico strategy for identifying small molecules that inhibit HIV-1 entry and infection of host cells. The strategy is based on the identification of an allosteric relationship between the GSL binding at the V3 loop tip and the chemokine receptor binding site at the V3 loop base whereby binding within the GSL binding site restricts the availability of the chemokine receptor binding site (illustrated in FIG. 3) by affecting the change in V3 loop order. Although, the V3 loop of gp120 is highly variable, it was found that the GSL binding site at the tip is largely conserved in all (more than 500 sequences) gp120s (see FIG. 4 and FIG. 5). Thus, the GSL binding site is a potential new target for anti-HIV drug development. An in si/co strategy is provided herein, for screening small molecule for identifying small molecules that are likely to bind at the base of gp120 V3 loop with high affinity and potential disrupt this N-terminal CKR-gp120 V3 interaction and thereby prevent V3 loop stem conformational change to inhibit HIV-1 entry and infection of host cells. Moreover, GSL binding within the V3 loop tip GSL binding site may similarly prevent V3 loop stem conformational change to prevent chemokine coreceptor binding. In addition GSL binding may directly block the CCR5 extracellular loop 2 binding to the V3 loop tip. In silico design strategies were utilized to identify small molecules with high affinity for these sites.

FIG. 3 illustrates AdamantylGSLs inhibition of gp120 binding to CD4/CCR5 transfected U87 cells. Cell bound R5 gp120 was detected by western blot. AdamantylGb₃ and adamantylGalCer pretreatment of R5 gp120 reduces its binding to CCR5 of cells demonstrating that binding the GSL binding site at the tip of the V3 loop does indeed prevent gp120-chemokine receptor binding. Lanes 1: cells alone; Cells treated with 2: 20 μg/mL gp120; 3: 20 μg/mL gp120+20 μM adaGb Gb₃; 4: 20 μg/mL gp120+50 μM ada Gb₃; 5: 20 μg/mL gp120+150 μM ada Gb₃; 6: 20 μg/mL gp120+5 μg/mL solCD4; 7: 20 μg/mL gp120+5 μg/mL solCD4+20 μM ada Gb₃; 8: 20 μg/mL gp120+5 μg/mL solCD4+50 μM ada Gb₃; 9: 20 μg/mL gp120+5 μg/mL solCD4+150 μM ada Gb₃; 10: 20 μg/mL gp120+50 μM.

FIG. 4 illustrates a mapping of variations in the V3 loop sequence. The amino acid/number proximal to the 3D structure designates the name and position of the amino acid in the V3 loop; the distal amino acids designated in parentheses (lighter font, if present) indicate the amino acid variation in CCR5; the amino acids shown in the intermediate/middle position designated in parentheses (darker font, if present) indicate the amino acid variation in CXCR4. FIG. 5 illustrates a mapping of the V3 loop sequence, in particular, note GPGRAF at positions 312-317, and GDIR at 324-327. The 35 amino acid sequence of the V3 loop, is known, and numbering convention used herein are those of, for example, NCBI (National Centre for Biotechnology Information, Bethesda, Md.).

GP120—Co-Receptor Interaction-Based Pharmacophore:

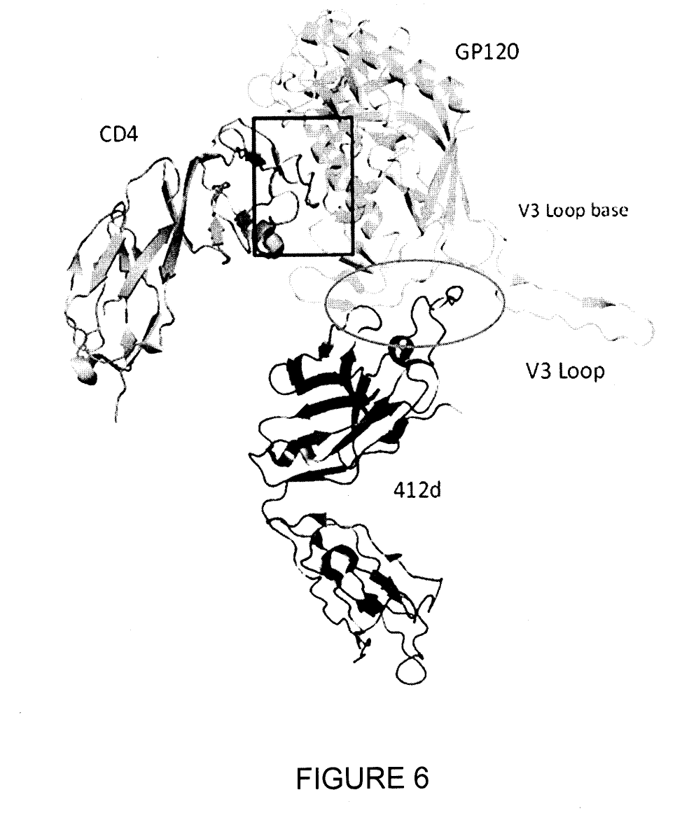

The proposed CCR5 binding site at the base of the V3 loop of gp120 was utilized for structure-based pharmacophore modeling. Interaction of sulfotyrosines (Tyr-Arg-Asp-Tyr amino acids) bearing region of 412d with base of V3 loop was used as template for gp120 interactions with co-receptor (PDB entry: 2QAD). The HIV gp 120 protein crystal structure (PDB ID: 2QAD) with complete V3 loop is used in this study. The Uniprot ID of GP120 (V3 loop) is P35961. This disulfotyrosine peptide mimics the N terminal disulfotyrosine peptide of CCR5. The key interactions observed between gp120 and CCR5 (which were conserved in the >500 X4, R5 and dual tropic gp120 V3 loop structures) were converted into 3D pharmacophore features. These features were combined to generate a set of 3D pharmacophore models (see FIG. 6). These models were used to retrieve novel compounds with diverse chemical scaffolds from databases of commercially available small-molecules. The predicted bound conformations of the selected compounds are analyzed (binding pose and score) and compounds prioritized. Lipinski's property, and toxicity filters were used to select molecules with lead-like properties. The compounds with reasonable binding affinity to gp120 are considered for synthesis and screening.

FIG. 6 illustrates crystal structure of CD4 induced gp120 complex with 412d. The region under the oval shows proposed site for gp120 interaction with co-receptors. The analysis of the co-crystal structure of CD4-induced gp120 complexed with antibody 412d bearing sulfated tyrosine residues revealed interaction of the O-sulfated tyrosines with the V3 loop base of gp120 (region encircled within the oval). To facilitate the binding of the two sulfotyrosines of 412d, the stem structure of the V3 loop becomes more ordered forming a hairpin. The observed interactions between O-sulfated tyrosines of 412d with the V3 loop base of gp120 mimic a co-receptor interaction with gp120. NMR studies of the N-terminal peptide (2-25) of CCR5, containing the two required tyrosine sulfates (Y10, Y14), show the formation of an ordered ahelical peptide (aa7-15) on binding gp120. This helical peptide was docked into the tyrosine sulfate binding site defined at the base of the V3 loop of the gp120-412d crystal complex to identify V3 loop residues interacting with CCR5. The N-terminal helical peptide aligns parallel to the V3 loop hairpin, interacting with this stem structure, which allows a second interaction whereby the CCR5 extracellular loop 2 binds to the V3 loop tip.

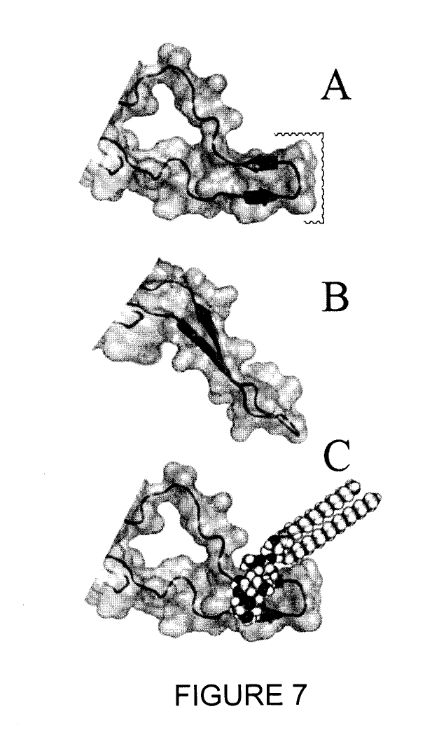

FIG. 7 illustrates a conformational change in gp120 induced by CCR5 binding and relation to GSL binding. The NMR structure of the V3 loop alone (A) or with bound CCR5 N terminus (B) is shown according to Huang et al., 2007. The amino acids of the GSL binding site at the loop apex are boxed in (A). The V3 loop, initially disorganized, becomes more rigid (beta-hairpin) on binding of the CCR5 N terminus to the V3 loop base. Gb₃ has been arbitrarily placed and oriented with its glucose moiety stacked over the phenylalanine of the CCR5 unbound loop (C) to illustrate the potential of GSL binding to affect this V3 loop conformational change.

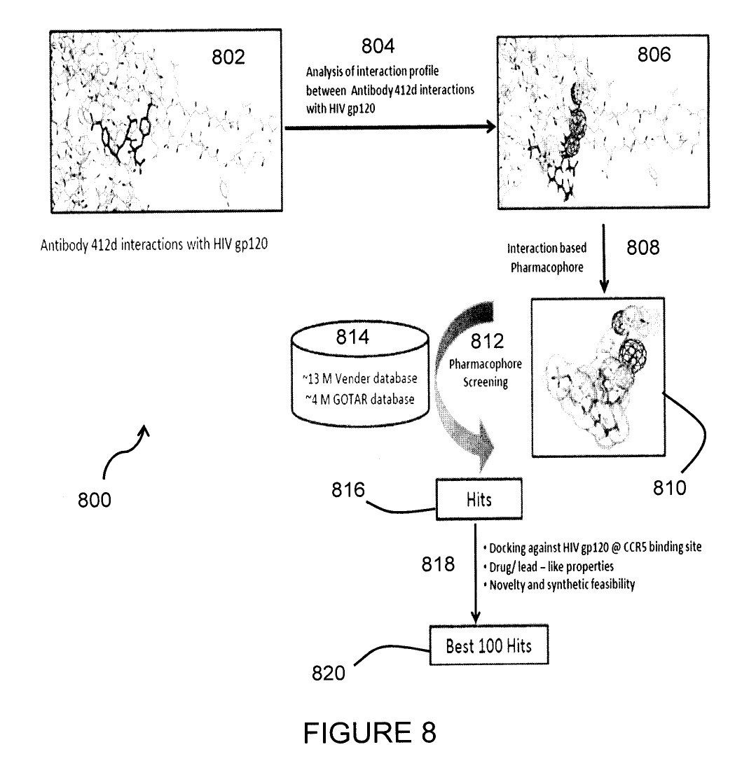

FIG. 8 illustrates the virtual screening protocol (800) using a pharmacophore model based on HIV-1 gp120 interaction with antibody 412d. An antibody 412d interaction with HIV gp120 is observed (802). Analysis (804) of the interaction profile between 412d with HIV gp120 is undertaken and modeled in silico (806), and the interaction analysis (808) serves as the basis for the development of a model pharmacophore (810). Upon modeling, compound screening (812) is undertaken, using databases (814) of molecules, for a similar pharmacophore that may suffice as a functionally corresponding pharmacophore, bearing similar properties as the model. Hits (816) in the database are docked against HIV gp120 at the CCRS binding site, and assessed (818) for docking ability. Drug leads are selected and assessed for novelty and synthetic feasibility. The top hits, for example the best 100 hits (820), can be assessed further in vitro.

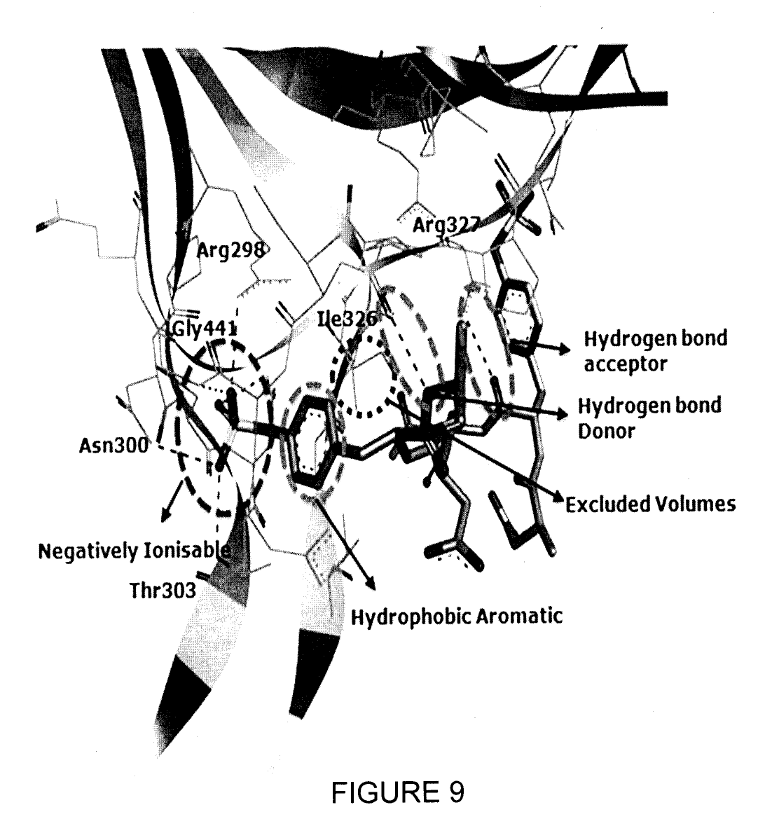

The key interactions observed between HIV gp120 and antibody 412d are converted into 3D pharmacophore features. A pharmacophore model was generated that includes of a negatively ionizable, a hydrophobic aromatic, two hydrogen bond donors, two hydrogen bond acceptors, and an aromactic ring feature. A negatively ionizable feature was added to represent 412d sulfotyrosine of Tyr100c charge-charge and hydrogen bonding interactions with gp120 V3 loop amino acid residues, Asn300, Gly441, Thr303, Arg298, Asn302. Å hydrophobic aromatic feature was also added, and considered to represent the phenyl group of 412d Tyr100c with gp120. Å hydrogen bond donor feature was included to represent backbone NH interaction of 412d Ala100D, the backbone carbonyl of residue Pro438 (gp120). Another hydrogen bond donor feature was considered to represent 412d Asn100 interaction with Gly324. Å hydrogen bond acceptor feature from the side chain of the carbonyl group of 412d Asn100 was considered where it makes a hydrogen bonding interaction with backbone NH of amino acid 11e326. Å hydrogen bond acceptor from main chain carbonyl group of 412d Tyr100 was considered, as it forms a hydrogen bond interaction with Arg327. Å ring aromatic feature was added to represent the interaction of 412d Tyr100 phenyl ring with Arg327. Å defined number of excluded volumes were added, centrally in the model, to avoid unfavorable interactions between potential binders and gp120 V3 base residues. A pharmacophore model showing key inter-feature distances is generally illustrated in FIG. 9, and is outlined in detail in FIG. 2.

In FIG. 9 and FIG. 2, key features of pharmacophore-1 are shown, representing key interactions between gp120 and 412d antibody. The model of pharmacophore-1 includes four features, namely: a negatively ionizable feature (upper left), a hydrophobic aromatic (below the negatively ionizabole portion, on the left), a hydrogen bond acceptor (lower right), and a hydrogen bond donor (upper right) feature.

The negatively ionizable feature represents salt-bridging and hydrogen bonding interactions between 412d sulfotyrosine of Tyr100c and the gp120 V3 loop amino acid residues: Asn300, Gly441, Thr303, Arg298 and Asn302.

The hydrophobic aromatic feature represents interaction between the phenyl group of 412d Tyr100c and gp120.

The hydrogen bond donor feature represents interaction between backbone NH of 412d Ala100D and the backbone carbonyl of residue Pro438 from gp120.

The hydrogen bond acceptor feature represents interaction between the main chain carbonyl group of 412d Tyr100 and with residue Arg327 of gp120.

The excluded volumes (central spheres of FIG. 2) are added to the model to avoid unfavorable interactions between potential binders and residues of gp120 V3 base.

The inter feature distance between negatively ionizable and hydrophobic aromatic feature is 4.094 Å in FIG. 2. A distance of from about 3 to about 5 Å or a distance of from about 3.5 to about 4.5 Å could result in a pharmacophore that functionally corresponds with pharmacophore-I.

The inter feature distance between hydrophobic aromatic and hydrogen bond acceptor feature is 8.987 Å in FIG. 2. A distance of from about 7 to about 11 Å or a distance of from about 8 to about 10 Å could result in a pharmacophore that functionally corresponds with pharmacophore-I.

The inter feature distance between negatively ionizable and hydrogen bond donor feature is 9.386 Å in FIG. 2. A distance of from about 7 to about 11 Å or a distance of from about 8 to about 19 Å could result in a pharmacophore that functionally corresponds with pharmacophore-I.

The hydrogen bond donor and acceptor features are separated by an inter feature distance of 6.188 Å in FIG. 2. A distance of from about 4 to about 8 Å or a distance of from about 5 to about 7 Å could result in a pharmacophore that functionally corresponds with pharmacophore-I.

The pharmacophore-1 model is used for virtual screening of small molecule databases for methods provided herein. Selected molecules were screened in biological assays as described herein.

Based upon the information show in FIG. 2 and in FIG. 9, pharmacophore-1A can be expressed as follows: (NI-AR)-EV-(HDₙ-HAₘ) (pharmacophore-1A), wherein: NI is a negatively ionizable feature; AR is a hydrophobic aromatic ring feature; EV is an excluded volume of at least 3 Å; HD is a hydrogen bond donor feature, wherein n is 1 or 2; HA is a hydrogen bond acceptor feature, wherein m is 1 or 2; the inter-feature distance between NI and AR is from about 3.5 to 4.5 Å; and the inter-feature distance between HD and HA is from about 5 to about 7 Å. Such inter-feature distances are expressed from the centre (or centroid) of the feature indicated. Excluded volumes (EV) represent an area in the pharmacophore that is not supposed to be occupied by any part of the structure.

Expressed another way, the pharmacophore may be described according to 3D co-ordinates in an X-Y-Z plane, for example some very specific co-ordingate representing FIG. 2 are as follows (X/Y/Z): negatively ionisable feature: (6.759/−3.517/63.036); hydrophobic aromatic feature: (3.225/−3.827/64.129); hydrogen bond acceptor feature: (0.69/−6.126/72.264); and hydrogen bond donor feature: (5.596/−9.532/68.57). Alternatively, the pharmacophore could be characterized as: negatively ionisable feature: (6 to 7/−3 to −4/62 to 65); hydrophobic aromatic feature: (3 to 4/−3 to −4/63 to 66); hydrogen bond acceptor feature: (0.5 to 0.8/−5 to −7/70 to 75); and hydrogen bond donor feature: (5 to 6/−8 to −11/66 to 70). Note that locations and/or distances are expressed from the centroid of a feature. Further, negatively ionizable and hydrophobic aromatic features are scalar with a radius of about 1.6 Å which reflects the tolerance or influence of the feature around the centroid. Hydrogen bond donor and hydrogen bond acceptor features are vector based and the volume of such a feature can indicate the tolerance or influence of the feature around the centroid (for example, an inner sphere with radius of 1.6 Å and outer sphere with 2.2 A). Excluded volume features are depicted based on a radius from centre, for example with a radius of 1.2 Å.

The pharmacophore may be represented with the formula of pharmacophore-1B as follows:

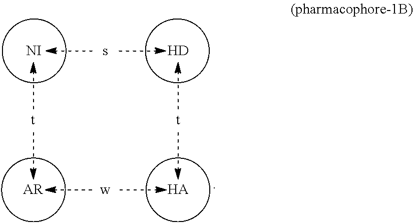

In this representation, NI is a negatively ionizable feature; AR is a hydrophobic aromatic ring feature; HD is a hydrogen bond donor feature, HA is a hydrogen bond acceptor feature, the inter-feature distance “t” between NI and AR is from about 3.5 to 4.5 Å; the inter-feature distance “w” between AR and HA is from about 8 to about 10 Å; and the inter-feature distance “s” between NI and HD is from about 8 to 10 Å; and the inter-feature distance “r” between HD and HA is from about 5 to about 7 Å. EV is not shown in this depiction, but an excluded volume may be assumed to be present in the central portion of the square formed by the 4 feature shown. An excluded volume permitting a distance of at least 3 Å between the right-side and left-side features of pharmacophore-1B is assumed. Inter-feature distances are expressed from the centre (or centroid) of the feature indicated. The excluded volumes (EV) represent an area in the pharmacophore that is not supposed to be occupied by any part of the structure.

A search using pharmacophore models retrieved a number of molecules from small molecules databases. Selected molecules were screened in biological assay.

Pharmaceutically acceptable salts of the compounds provided are also encompassed. Compounds provided herein can possess a sufficiently acidic, a sufficiently basic, or both functional groups, and accordingly react with a number of organic and inorganic bases, and organic and inorganic acids, to form pharmaceutically acceptable salts.

Acids commonly employed to form acid addition salts are inorganic acids such as hydrochloric acid, hydrobromic acid, hydroiodic acid, sulphuric acid, and phosphoric acid, and organic acids such as p-toluenesulphonic acid, methanesulphonic acid, oxalic acid, p-bromophenylsulphonic acid, carbonic acid, succinic acid, citric acid, benzoic acid, and acetic acid. Examples of such pharmaceutically acceptable salts are the sulphate, pyrosulphate, bisulphate, sulphite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulphonate, phenylacetate, phenylpropionate, phenylbutyrate, citrate, lactate, gamma-hydroxybutyrate, glycolate, tartrate, methanesulphonate, propanesulphonate, naphthalene-I-sulfonate, napththalene-2-sulfonate, and mandelate. Preferred pharmaceutically acceptable acid addition salts are those formed with mineral acids such as hydrochloric acid and hydrobromic acid, and those formed with organic acids such as maleic acid and methanesulphonic acid.

Salts of amine groups may also comprise quarternary ammonium salts in which the amino nitrogen carries a suitable organic group such as an alkyl, lower alkenyl, substituted lower alkenyl, lower alkynyl, substituted lower alkynyl, or aralkyl moiety.

Base addition salts include those derived from inorganic bases, such as ammonium or alkali or alkaline earth metal hydroxides, carbonates, and bicarbonates. Bases useful in preparing the salts include sodium hydroxide, potassium hydroxide, ammonium hydroxide, potassium carbonate, sodium carbonate, sodium bicarbonate, potassium bicarbonate, calcium hydroxide, and calcium carbonate.

One skilled in the art will understand that the particular counterion forming a part of a salt is usually not of a critical nature, so long as the salt as a whole is pharmacologically acceptable and as long as the counterion does not contribute undesired qualities to the salt as a whole. Pharmaceutically acceptable solvates of a compound of Formula I are also encompassed herein. Many of the compounds of Formula I can combine with solvents such as water, methanol, ethanol and acetonitrile to form pharmaceutically acceptable solvates such as the corresponding hydrate, methanolate, ethanolate and acetonitrilate.

Pharmaceutical Compositions

The compounds described herein are typically formulated prior to administration. Pharmaceutical compositions comprising one or more compounds of Formula I and a pharmaceutically acceptable carrier, diluent, or excipient are also encompassed herein. The pharmaceutical compositions are prepared by known procedures using well-known and readily available ingredients.

Compounds of the general Formula I or salts thereof, or pharmaceutical compositions comprising the compounds or salts may be administered orally, topically, parenterally, by inhalation or spray, or rectally in dosage unit formulations containing conventional non-toxic pharmaceutically acceptable carriers, adjuvants and vehicles. In the usual course of therapy, the active compound is incorporated into an acceptable vehicle to form a composition for topical administration to the affected area, such as hydrophobic or hydrophilic creams or lotions, or into a form suitable for oral, rectal or parenteral administration, such as syrups, elixirs, tablets, troches, lozenges, hard or soft capsules, pills, suppositories, oily or aqueous suspensions, dispersible powders or granules, emulsions, injectables, or solutions. The term parenteral as used herein includes subcutaneous injections, intradermal, intra-articular, intravenous, intramuscular, intravascular, intrasternal, intrathecal injection or infusion techniques.

Compositions intended for oral use may be prepared in either solid or fluid unit dosage forms. Fluid unit dosage form can be prepared according to procedures known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations. An elixir is prepared by using a hydroalcoholic (e. g., ethanol) vehicle with suitable sweeteners such as sugar and saccharin, together with an aromatic flavoring agent. Suspensions can be prepared with an aqueous vehicle with the aid of a suspending agent such as acacia, tragacanth, and methylcellulose.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate : granulating and disintegrating agents for example, corn starch, or alginic acid: binding agents, for example starch, gelatin or acacia, and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions contain active materials in admixture with excipients suitable for the manufacture of aqueous suspensions. Such excipients are suspending agents, for example sodium carboxylmethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl-p-hydroxy benzoate, one or more colouring agents, one or more flavouring agents or one or more sweetening agents, such as sucrose or saccharin.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions provided herein may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

The pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables. Adjuvants such as local anaesthetics, preservatives and buffering agents can also be included in the injectable solution or suspension.

The compound(s) of the general Formula I may be administered, together or separately, in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Other pharmaceutical compositions and methods of preparing pharmaceutical compositions are known in the art.

Administration of Compounds

Compounds described herein, such as those of Formula I, Compounds #1-#56, or salts and functional derivatives thereof may be administered to a subject by a variety of routes, for example, the compounds may be administered orally, topically, parenterally, by inhalation or spray, or rectally in dosage unit formulations. In one embodiment, the compounds are administered systemically to a subject, for example, by bolus injection or continuous infusion into a subject's bloodstream or by oral administration.

The dosage to be administered is not subject to defined limits, but it will usually be an effective amount. It will usually be the equivalent, on a molar basis of the pharmacologically active free form produced from a dosage formulation upon the metabolic release of the active free drug to achieve its desired pharmacological and physiological effects. The compositions may be formulated in a unit dosage form.

However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended as limiting. In some instances dosage levels below the lower limit of the aforesaid range may be more than adequate, while in other cases still larger doses may be employed without causing harmful side effects, for example, by first dividing the larger dose into several smaller doses for administration throughout the day.

The effective infection inhibiting dose may be administered at regular intervals. The effective infection inhibiting dose may be administered at one or more predefined intervals. The predefined interval may be at least once weekly, every two to four weeks, every two weeks, every three weeks, every four weeks, at least once monthly, every six weeks, or every eight weeks.

The instant methods may further comprise administering to the subject at least one additional antiretroviral agent effective against HIV.

Kits

Therapeutic kits are provided herein, containing one or more compounds of Formula I for use in the treatment of HIV infection. The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceutical or biological products, which notice reflects approval by the agency of manufacture, for use or sale for human or animal administration.

When the components of the kit are provided in one or more liquid solutions, the liquid solution can be an aqueous solution, for example a sterile aqueous solution. For in vivo use, the compounds may be formulated into a pharmaceutically acceptable injectable composition. In this case the container means may itself be an inhalant, syringe, pipette, eye dropper, or other such like apparatus, from which the formulation may be applied to an infected area of the subject, such as the lungs, injected into a subject, or even applied to and mixed with the other components of the kit.

The following examples are set forth for better understanding. It should be understood that these examples are for illustrative purposes only, and are not intended to be limiting.

### EXAMPLE 1

Protocol 1—Determining Efficacy of Compounds to Inhibit HIV-1 in Jurkat T-Cells

The following protocol was used to determine the efficacy of certain selected compounds having the structure according to Formula I in inhibiting HIV-1.

HIV-1IIIB virus (X4 tropism) was pretreated with a control solution of DMSO or a given compound dissolved in DMSO to a final concentration of 100 μM for 1 hr at 37° C. Pretreated HIV-1 virus was then added (100 μl of 0.1 MOI) to 0.5×10⁶ Jurkat (JKT C) T-cells in 100 μl RPMI media and incubated at 37° C. for an additional 1 hr with occasional mixing.

After 1 hour, the cells were centrifuged at 1000 rpm for 5 minutes and the cell pellet washed wash thrice with 2 ml Ca²⁺ Mg²⁺ free PBS. After the last wash, the cells were resuspended in medium and transferred to 12-well tissue culture plates and placed at 37° C.

On days 2 and 5 of culture, 0.5 ml aliquots of culture supernatant is collected and replaced with 0.5 ml of fresh medium. Aliquots are stored at −80° C. until ready for p24gag ELISA. ELISA for p24gag was done using ELISA plates from Zeptometrix using a standard protocol.

Results obtained with Protocol 1, in which compounds #1, #2, #3, #25, #27, #29, #32, #36, #37, #38 and #39 were tested, are shown below. The following inhibition results are expressed as percent inhibitory activity in the presence of a given concentration of the specified compound (concentration shown in parentheses):

#1: 3% (100 μM);

#2: 88% (100 μM);

#3: 71% (100 μM);

#25: 18% (100 μM);

#27: 20% (100 μM):

#29: 75% (20 μM);

#32: 80% (20 μM);

#36: 50% (20 μM);

#37: 45% (100 μM);

#38: 44% (100 μM); and

#39: 32% (100 μM).

### EXAMPLE 2

Protocol 2—Determining Efficacy of Compounds to Inhibit HIV-1 Using Peripheral Blood Mononucleated Cells (PBMC)

The following protocol was used to determine the efficacy of certain selected compounds having the structure according to Formula I for inhibiting HIV-1.

The protocol involves PBMCs which were purified by centrifugation at 500 g using Ficoll-Paque™ reagent. The cells were re-suspended in RPMI (10% FBS, 2 ug/ml PHA, 20 U/ml of IL-2) and incubated at 37° C. in 5% CO₂ for 3 days.

HIV-1 BaL virus (R5 tropism) was pre-treated with a control solution of DMSO or a given compound dissolved in DMSO to a final concentration of 20-100 μM for 1 hour at 37° C. Pretreated HIV-1 viruses were then added (100 μl of 0.1 MOI) to 5×10⁵ PBMCs in 100 μl RPMI media and then incubated at 37° C. for an additional 1 hour with occasional mixing. After 1 hour, the cells were centrifuged at 1000 rpm for 5 minutes and the cell pellet washed wash thrice with 2 ml Ca²⁺ Mg²⁺ free PBS.

After the last wash, the cells are re-suspended in medium and transferred to 12-well tissue culture plates and place at 37° C.

On day 6 of culture, 0.5 ml aliquots of culture supernatant were collected. Aliquots were stored at −80° C. until ready for p24gag ELISA. ELISA for p24gag was done using ELISA plates from Zeptometrix using a standard protocol.

In results obtained with Protocol 2, compounds #2, #3, and #21 were tested for inhibitory activity. The results are expressed as percent inhibitory activity in the presence of a given concentration of the compound (concentration shown in parentheses):

#2: 30% (100 μM);

#3: 34% (100 μM); and

#21: 40% (100 μM).

### EXAMPLE 3

Protocol 3—Determining Efficacy of Compounds to Inhibit HIV-1 Using TZM-bl Cells

The following protocol was used to determine the efficacy of certain selected compounds having the structure according to Formula I for inhibiting HIV-1.

HIV-BaL virus (R5 tropism) and HIV-1 IIIb virus (X4 tropism) were pretreated with a given compound dissolved in DMSO to a final concentration of 100-200 μM for 1 hr at 37° C. in 96 well tissue culture plates. The TZM-bl HIV reporter cell line (1×10⁴/well) in 100 ul DMEM is then added to each well and the 96 well tissue culture plates are incubated at 37° C. in 5% CO₂ incubator.

TZM-bl cells were obtained from the NIH AIDS Reagent Program (Germantown Md., USA). TZM-bl, previously designated JC53-bl (clone 13) is a HeLa cell line. The parental cell line (JC.53) stably expresses large amounts of CD4 and CCR5. The TZM-bl cell line was generated from JC.53 cells by introducing separate integrated copies of the luciferase and ß-galactosidase genes under control of the HIV-1 promoter. The TZM-bl cell line is highly sensitive to infection with diverse isolates of HIV-1. The TZM-bl indicator cell line enables simple and quantitative analysis of HIV using either ß-gal or luciferase as a reporter. It is maximally sensitive to HIV infection by including DEAE-dextran in the infection medium. The ß-gal and luciferase genes are also induced by HIV-2 infection. Further information about TZM-bl cells can be found in U.S. Pat. No. 6,797,462, issued to Tranzyme Pharma.

After 48 hours, half of the medium was removed and replaced with commercially available reagents that contain lysing buffer and substrate for luciferase. After 2 minutes of lysis and incubation, the luciferase activity of the TZM-bl cell is read using a commercial luminometer. The readout is translated as relative light units (RLU).

In results obtained with Protocol 3, compounds #32, #34, #38, #39, #40, #41, #42, #43, and #47 were tested for inhibitory activity. The following results show percent inhibitory activity in the presence of a given concentration of the compound (concentration shown in parentheses) for inhibitory activity against R5 and inhibitory activity against X4, respectively:

| | | |
|---|---|---|
| #32 | 94% (200 μM) | 61% (200 μM); |
| #34 | 61% (200 μM) | 62% (200 μM); |
| #38 | 73% (100 μM) | 69% (100 μM); |
| #39 | 15% (100 μM) | 15% (100 μM); |
| #40 | 71% (100 μM) | 57% (100 μM); |
| #41 | 14% (100 μM) | 10% (100 μM); |
| #42 | 25% (100 μM) | 25% (100 μM); |
| #43 | 12% (100 μM) | 16% (100 μM); and |
| #47 | 27% (100 μM) | 29% (100 μM). |

### EXAMPLE 4

Protocol 4—Determining Efficacy of Compounds to Inhibit HIV-1 Using U87. CD4. CCR5 Cells

The following protocol was used to determine the efficacy of certain selected compounds having the structure according to Formula I for inhibiting HIV-1.

U87.CD4.CCR5 cell lines (5×10⁴/well) were seeded in 24 well plates overnight. HIV-BaL virus (R5 tropism) was pretreated with a control solution of DMSO or a given compound dissolved in DMSO to a final concentration of 20-200 μM for 1 hr at 37° C.

Pretreated HIV-1 virus was then added (100 μl of 0.1 MOI) to pre-plated U87.CD4.CCR5 cells in 200 μl DMEM media and then incubated at 37° C. for an additional 1 hour with occasional mixing.

After 1 hour, the cells in each well were washed with 1 ml Ca²⁺ Mg²⁺ free PBS three times. After the last wash, 1 ml of DMEM was added to each well. On day 4 post-infection, 0.2 ml aliquots of culture supernatant was collected. Aliquots were stored at −80° C. until ready for p24gag ELISA. ELISA for p24gag was done using ELISA plates from Zeptometrix using a standard protocol.

Compounds #28, #29, #30, #31, #33, #34, #35, #36, #37, and #44 were tested for inhibitory activity using Protocol 4. The results shown below are expressed as percent inhibitory activity in the presence of a given concentration of the compound (concentration shown in parentheses):

| | |
|---|---|
| #28 | 27% (20 μM); |
| #29 | 24% (20 μM); |
| #30 | 19% (20 μM); |
| #31 | 21% (20 μM); |
| #33 | 27% (20 μM); |
| #34 | 33% (20 μM); |
| #35 | 27% (20 μM); |
| #36 | 7% (20 μM); |
| #37 | 28% (200 μM); and |
| #44 | 10% (20 μM). |

### EXAMPLE 5

Testing Specific Compounds (#32 and #38) Using Human Blood Cells

In this Example, Compounds #32 and #38 were tested using human peripheral blood mononuclear cells (PBMC). The protocol used is the same as that of Example 2—Protocol 2, delineated in detail, above.

Methods. Briefly, 5×10⁵ activated PBMCs were used per well in a 12-well tissue culture plate. The PBMCs were infected with HIV BaL for 1 hour and washed 2× with RPMI medium. The PBMCs were then mixed with compound #32 or #38 at concentrations of 6.0 μM, 25 μM or 100 μM, with solutions containing 20U/m1 of IL-2.

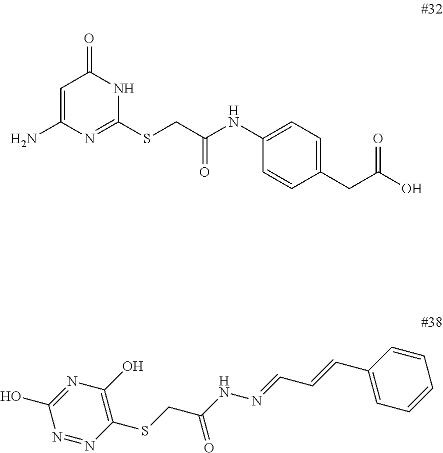 [SMILES: Nc1cc(=O)[nH]c(SCC(=O)Nc2ccc(CC(=O)O)cc2)n1 ; O=C(CSc1nnc(O)nc1O)N/N=C/C=C/c1ccccc1]

At day 3-post infection, 500 μl of fresh medium containing appropriate concentration of compounds were added. The supernatants were harvested at day 8. The level of HIV infection was determined as described in protocol 2.

The following depiction of Compound #32-A shows, with dashed circles, regions attributable to the following features (from left to right): HD (hydrogen bond donor); HA (hydrogen bond acceptor); AR (hydrophobic aromatic); NI (negatively ionizable).

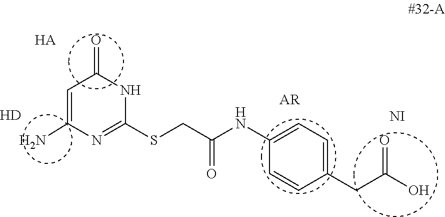

Results. The % HIV inhibitory activity was evaluated for each treatment, and results are as follows, with concentrations of compounds #32 or #38 shown in parentheses.

#32 82% (100μM); 65% (25μM); 54% (6.0μM).

#38 100% (100μM); 90% (25μM); 70% (6.0μM).

These results indicate inhibitory activity for both compounds, even at the lower concentrations of 6 μM.

### EXAMPLE 6

Testing Compounds #48, #49, #50 and #51 Derived from Compound #38

In this Example, four compounds considered to be functional derivatives of compound #38 were tested for HIV inhibitory activity.

Methods. U87.CD4.CCR5 cells (2.5×10⁴/well) were seeded overnight and then infected with a replication-deficient HIV strain containing a luciferase insertion in the nef gene (JR-FL-R5 virus). JR-FL-R5 virus was pre-incubated with each compound for 1 hour prior to infection. After two days, the cells were washed, lysed and measured for luciferase activity, which is indicative of viral integration and/or transcription. Compounds were tested in quadruplicates.

Results. The results of the testing protocol are reported below as % HIV inhibitory activity. Concentrations or the compounds are shown in parentheses.

Compound #32 showed 32% HIV inhibitory activity (2 μM).

Compound #38 showed 30% HIV inhibitory activity (2 μM).

Compound #48 showed 28% HIV inhibitory activity (0.05 μM).

Compound #49 showed 45% HIV inhibitory activity (0.05 μM).

Compound #50 showed 32% HIV inhibitory activity (0.05 μM).

Compound #51 showed 22% HIV inhibitory activity (0.05 μM).

These data illustrate that compounds #48, #49, #50 and #51 can inhibit HIV activity, even when used at lower concentrations that the compound #38, upon which the structures are based. Thus, compounds #48, #49, #50 and #51 can be said to be functional derivatives of compound #38.

### EXAMPLE 7

Further Compounds Structurally Derived from Compounds #37 and #38

In this Example, derivatives of Compound #37 and #38 are described, resulting in compounds #52, #53, #54, #55, and #56, considered to be functional derivatives of either compound #37 or #38.

Structural modeling data (not shown) was used to determine that compounds #52, #53, #54, #55, and #56 are structurally appropriate to function at the target of site from residue 324 to residue 327, referenced herein as: 324GDIR327. This site has not been previously recognized as a site of anti HIV drug vulnerability within the HIV Gp120 V3loop. This site, where the glycolipid binding site overlaps that of the chemokine receptor binding site (Lingwood & Branch, 2011) was targeted in a model based in part on a glycolipid HIV resistance factor described by Lund et al, (2009). The resulting model used in this example permitted formation of new functional derivative structures, based on compound #37 and/or #38, above, for formation of small molecule inhibitors capable of blocking the host cell entry of both R5 and X4 HIV strains.

Based on the in silico molecular docking of compounds #37 and #38 within the V3 loop, an additional chemical modification was undertaken, resulting in the additional binding to a conserved V3 loop sequence defined as the recognition peptide to a family of pan-HIV neutralizing antibodies. The inhibitor derived on this basis may block CD4-dependent gp120-chemokine receptor binding and/or interfere with gp120-chemokine receptor binding signal transduction mechanisms required for HIV entry into host cells, and may also target a further epitope crucial to the host cell entry of many HIV strains.

Additional chemical modifications may be effected on active inhibitors of HIV, so as to extend the degree of interaction within the base of the V3 loop to include the 324GDIR327 conserved amino acid sequence, bound by several broadly neutralizing anti-HIV monoclonal antibodies. The resulting small molecule inhibitors of HIV infection may simultaneously target three conserved sites of vulnerability within the crucial V3 loop of

GP120 between the V3 loop base, the V3 loop tip and the crucial conserved region of the V3 loop alpha helix. This triple mechanism of anti-HIV activity may effectively inhibit HIV infection, and can be useful in the field of anti-HIV therapy, particularly as entry inhibitors. Entry inhibitors are currently used as a mainstay against drug resistant HIV strains.

Thus, it may be said that the mechanism of action by which compounds #32, #37, #38, #52, #53, #54, #55, #56, salts thereof, and functional derivatives thereof can inhibit, treat or prevent HIV is in part through entry inhibition.

Compounds #52, #53, #54, #55, and #56 are shown below:

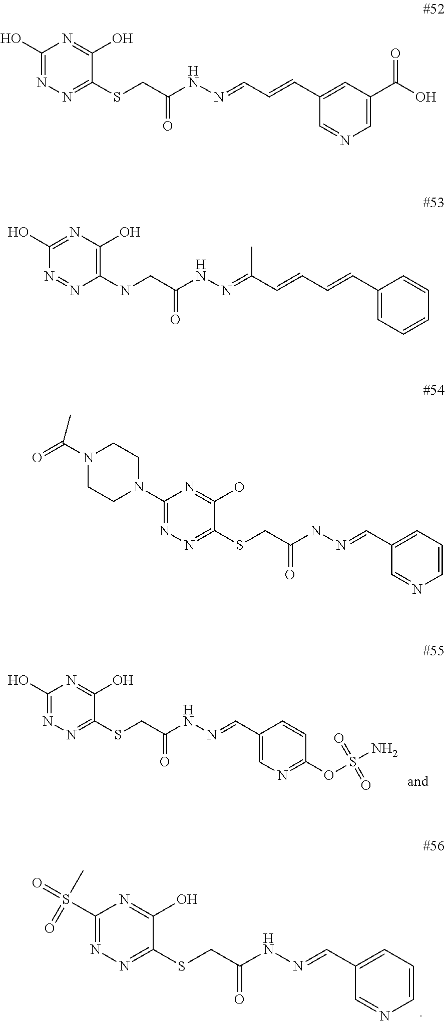 [SMILES: O=C(CSc1nnc(O)nc1O)N/N=C/C=C/c1cncc(C(=O)O)c1 ; CC(/C=C/C=C/c1ccccc1)=N\NC(=O)CNc1nnc(O)nc1O ; CC(=O)N1CCN(c2nnc(SCC(=O)N/N=C/c3cccnc3)c(O)n2)CC1 ; NS(=O)(=O)Oc1ccc(/C=N/NC(=O)CSc2nnc(O)nc2O)cn1 ; CS(=O)(=O)c1nnc(SCC(=O)N/N=C/c2cccnc2)c(O)n1]

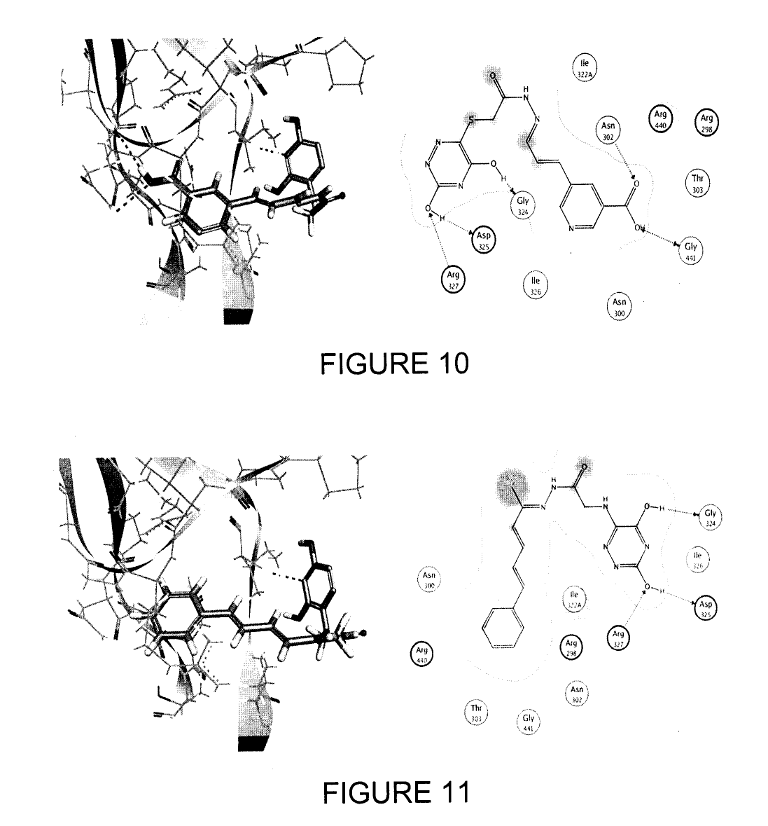

FIG. 10 shows GP-120 V3-loop interactions of compound #52. On the left side, the docking pose of compound #52 within the GP-120 V3-loop is modelled in 3-dimensions, showing interaction with certain amino acids of the V3-loop: Arg(R)327, Asp(D)325, Gly(G)324, Asn(N)302, and Gly(G)324. On the right side, a 2-dimensional image is provided, representing the interactions of these amino acids with the relevant portions of the compound. These depictions support the use of compound #52 as an inhibitor of HIV.

FIG. 11 shows GP-120 V3-loop interactions of compound #53. On the left side, the docking pose of compound #53 within the GP-120 V3-loop is modelled in 3-dimensions, showing interaction with certain amino acids of the V3-loop: Arg(R)327. Asp(D)325, and Gly(G)324. On the right side, a 2-dimensional image is provided, representing the interactions of these amino acids with the relevant portions of the compound. These depictions support the use of compound #53 as an inhibitor of HIV.

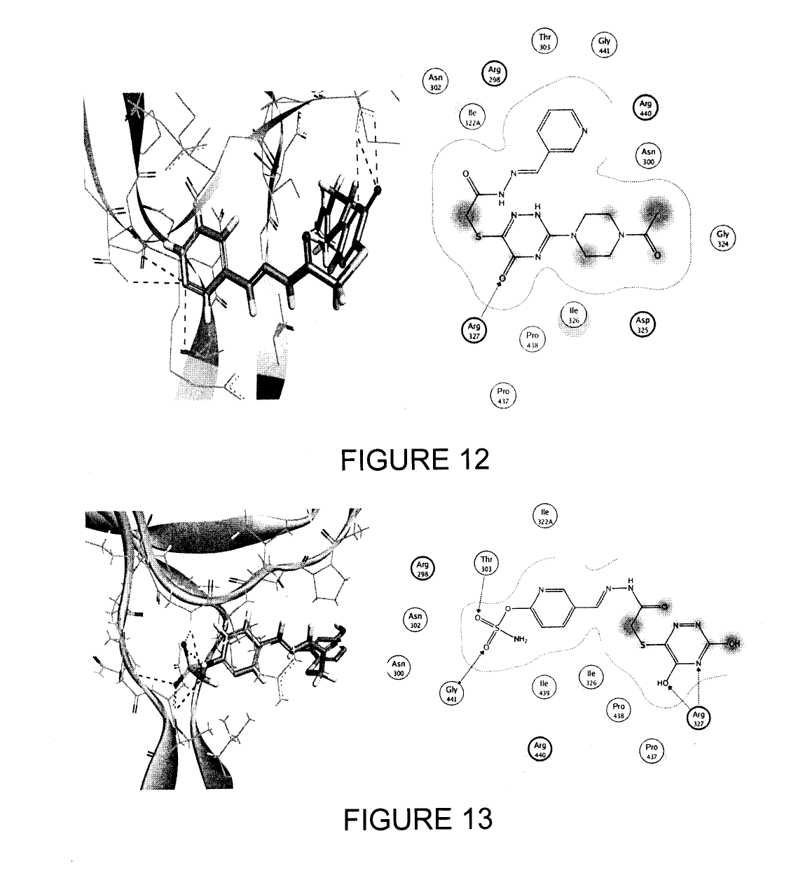

FIG. 12 shows GP-120 V3-loop interactions of compound #54. On the left side, the docking pose of compound #54 within the GP-120 V3-loop is modelled in 3-dimensions, showing interaction with Arg(R) 327 within the V3-loop. Further, the positions of Asp(D)325 and Ile(1)326 are illustrated. On the right side, a 2-dimensional image is provided, representing the interaction and/or positioning of certain amino acids with the relevant portions of the compound. These depictions support the use of compound #54 as an inhibitor of HIV.

FIG. 13 shows GP-120 V3-loop interactions of compound #55. On the left side, the docking pose of compound #55 within the GP-120 V3-loop is modelled in 3-dimensions, showing interaction with certain amino acids of the V3-loop: Arg(R)327 interaction is shown, as well as Thr(T)303, and Gly(G)441. On the right side, a 2-dimensional image is provided, representing the interactions of these amino acids with the relevant portions of the compound. These depictions support the use of compound #55 as an inhibitor of HIV.

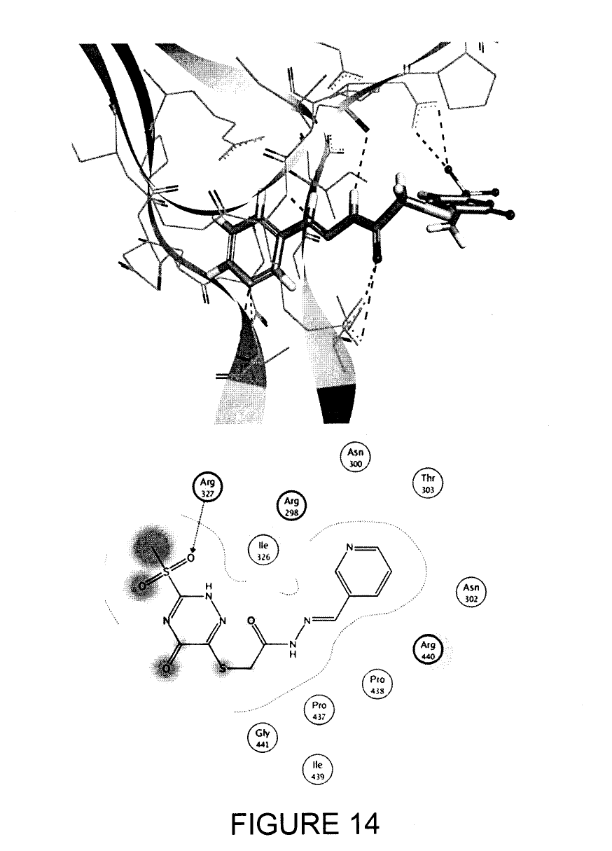

FIG. 14 shows GP-120 V3-loop interactions of compound #56. On the left side, the docking pose of compound #52 within the GP-120 V3-loop is modelled in 3-dimensions, showing interaction with the V3-loop, and specifically the positioning of Arg(R)327. On the right side, a 2-dimensional image is provided, representing the interactions or positioning of V3-loop amino acids with the relevant portions of the compound. These depictions support the use of compound #56 as an inhibitor of HIV.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope, which is defined solely by the claims appended hereto.

### REFERENCES

Andrianov, A. M., et al. (2011). “Discovery of Novel Promising Targets for Anti-AIDS Drug Developments by Computer Modeling: Application to the HIV-1 gp120 V3 Loop.” J Chem Inf Model 2011 Oct. 24;51(10):2760-7.

Delezay, O., et al. (1996). “SPC3, a V3 loop-derived synthetic peptide inhibitor of HIV-1 infection, binds to cell surface glycosphingolipids.” Biochemistry 35(49): 15663-15671.

Fantini, J., et al. (1993). “Infection of colonic epithelial cell lines by type 1 human immunodeficiency virus is associated with cell surface expression of galactosylceramide, a potential alternative gp120 receptor.” Proc Natl Acad Sci USA 90(7): 2700-2704.

Huang, C. C., et al. (2007). “Structures of the CCR5 N terminus and of a tyrosine-sulfated antibody with HIV-1 gp120 and CD4.” Science 317(5846): 1930-1934.

Joly, V., et al. (2010). “Enfuvirtide: from basic investigations to current clinical use.” Expert Opin Pharmacother 11(16): 2701-2713.

Liao, Z., et al. (2001). “Lipid rafts and HIV pathogenesis: host membrane cholesterol is required for infection by HIV type 1.” AIDS Res Hum Retroviruses 17(11): 1009-1019.

Lingwood C A, and Branch D R. Glycosphingolipids in HIV/AIDS: The Potential Therapeutic Application. InTech. (2011); Understanding HIV/AIDS Management and Care—Pandemic Approaches in the 21st Century: 239-80.

Lund, N., et al. (2006). “A novel soluble mimic of the glycolipid globotriaosylceramide inhibits HIV infection.” AIDS 20: 333-343.

Lund, N., et al. (2005). “Lack of Susceptibility of Cells from Patients with Fabry Disease to Infection with R5 Human Immunodeficiency Virus.” AIDS 19(14): 1543-1546.

Lund, N., et al. (2009). “The human Pk histo-blood group antigen provides protection against HIVinfection.” Blood 113(20): 4980-4991.

Mahfoud, R., et al. (2002). “Identification of a common sphingolipid-binding domain in Alzheimer, prion and HIV-1 proteins.” J Biol Chem 277: 11292-11296.

Parra, J., et al. (2011). “Clinical utility of maraviroc.” Clin Drug Investig 31(8): 527-542.

U.S. Pat. No. 6,797,462

The disclosure of each patent, publication, including any published patent application, and database entry referenced in this specification is hereby expressly incorporated by reference to the same extent as if each such individual patent, publication, or database entry was expressly and individually indicated to be incorporated by reference.

1. A compound of formula (I) 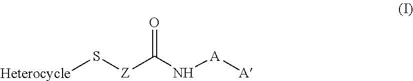
or a pharmaceutically acceptable salt thereof, for use in treating, inhibiting, or preventing HIV infection in a subject, wherein:
A is absent or is —N═CH—;
Z is —CH₂— or —CH₂—CH₂—;
A′ is C₆-C₁₀ aryl or C₆-C₁₀ heteroaryl, optionally substituted with one or more of C₁-C₆ alkyl, —OH, —O—C₁-C₆ alkyl, —OCX₃, halogen, —C(O)OR, —SO₂NR′R″, —NR′R″ or —O—(CH₂)₁₋₃—O— to form a bicyclic ring; wherein X is halogen, R is C₁-C₆ alkyl, R′ and R″ are independently H, C₁-C₆ alkyl, or R′ and R″ taken together with N form a C₃-C₇ heterocycle comprising one or more additional hetero atoms selected from N and O; and
heterocycle(s) are substituted or unsubstituted;
wherein the compound comprises a pharmacophore functionally corresponding with pharmacophore I of FIG. 2.

2. The compound of claim 1, wherein said aryl is phenyl or naphthyl, optionally substituted with one or more of said substituents.

3. (canceled)

4. The compound of claim 1, wherein said aryl is phenyl substituted with one or more substituents selected from the group consisting of methyl, ethyl, propyl, isopropyl, butyl, —F, Cl, —OH, —O-n-propyl, —O-isopropyl, —C(O)OH, —C(O)OCH₃, —C(O)OCH₂CH₃, —O—CF₃, —O—CCl₃, —SO₂NH₂, and —SO₂NR′R″ wherein R′ and R″ are H or taken together with N atom to form a five a six membered ring, wherein said 5— or 6-membered ring optionally having an oxygen atom in addition to the N atom.

5. The compound of claim 1, wherein said aryl is phenyl substituted with —O—CH₂—O— or —O—(CH₂)₂—O— to form: 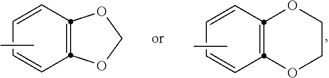
respectively.

6. The compound of claim 1, wherein said heteroaryl is a pyridinyl group.

7. The compound of claim 1, wherein said heterocycle is 5 or 6 membered heterocycle, comprising one or more ring nitrogen atoms, optionally substituted with one or more —OH, —NH₂ or oxo groups.

8. The compound of claim 1, wherein the pharmacophore comprises a negatively ionizable feature, a hydrophobic aromatic feature, a hydrogen bond acceptor feature, and a hydrogen bond donor feature.

9. The compound of claim 8, wherein the pharmacophore comprises a negatively ionizable feature, a hydrophobic aromatic feature, two hydrogen bond acceptor features, two hydrogen bond donor features, and a central excluded volume.

10. The compound of claim 8, wherein the pharmacophore comprises inter-feature distances of:
between the negatively ionizable feature and hydrophobic aromatic feature a distance of from about 3 to about 5 Å, or from about 3.5 to about 4.5 Å;
between the hydrophobic aromatic feature and the hydrogen bond acceptor feature a distance of from about 7 to about 11 Å, or from about 8 to about 10 Å;
between the negatively ionizable feature and the hydrogen bond donor feature a distance of from about 7 to about 11 Å, or from about 8 to about 10 Å; and
between the hydrogen bond donor feature and the hydrogen bond acceptor feature a distance of from about 4 to about 8 Å, or from about 5 to about 7 Å.

11. The compound of claim 1, wherein the pharmacophore comprises pharmacophore-1A:
(NI-AR)-EV-(HDₙ-HAₘ) (pharmacophore-1A)
wherein:
NI is a negatively ionizable feature;
AR is a hydrophobic aromatic ring feature;
EV is an excluded volume of at least 3 Å;
HD is a hydrogen bond donor feature, wherein n is 1 or 2;
HA is a hydrogen bond acceptor feature, wherein m is 1 or 2;
the inter-feature distance between NI and AR is from about 3.5 to 4.5 Å; and
the inter-feature distance between HD and HA is from about 5 to about 7 Å.

12. The compound of claim 1 for use in treating, inhibiting, or preventing HIV infection in a subject, wherein the compound is selected from the group consisting of 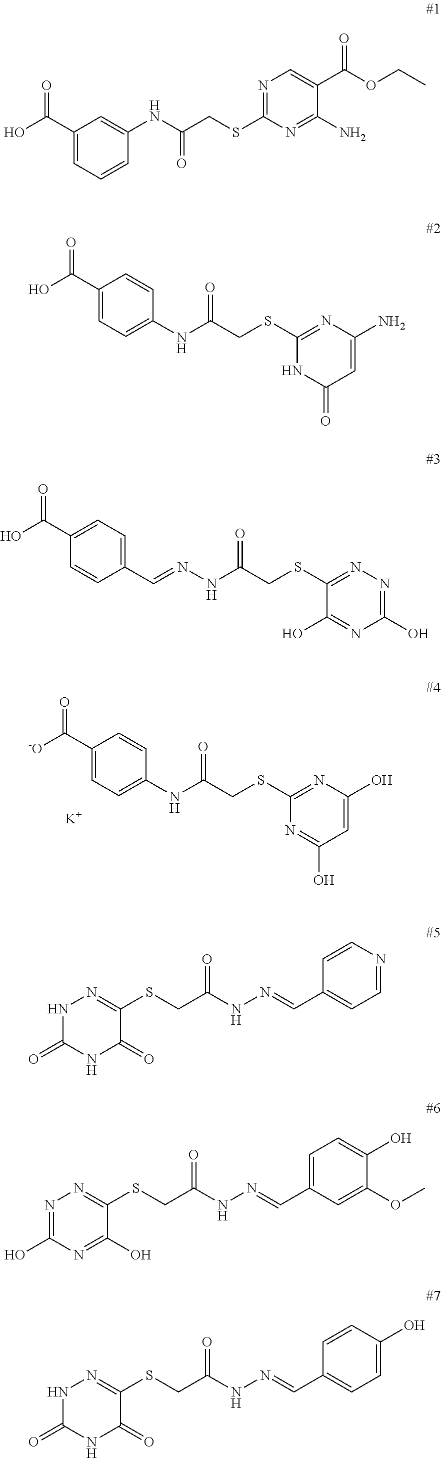 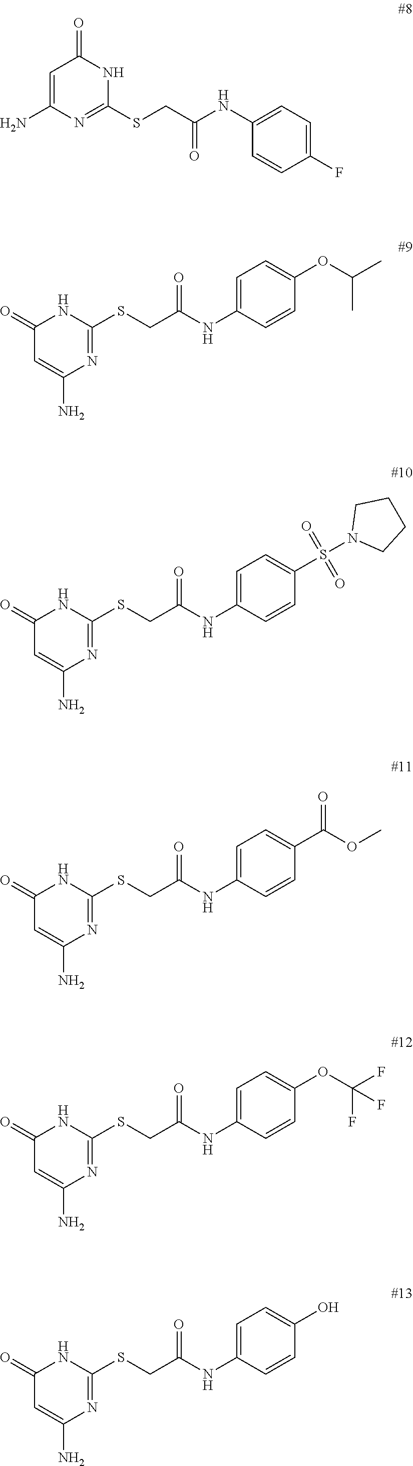 [SMILES: Nc1cc(=O)[nH]c(SCC(=O)Nc2ccc(F)cc2)n1 ; CC(C)Oc1ccc(NC(=O)CSc2nc(N)cc(=O)[nH]2)cc1 ; Nc1cc(=O)[nH]c(SCC(=O)Nc2ccc(S(=O)(=O)N3CCCC3)cc2)n1 ; COC(=O)c1ccc(NC(=O)CSc2nc(N)cc(=O)[nH]2)cc1 ; Nc1cc(=O)[nH]c(SCC(=O)Nc2ccc(OC(F)(F)F)cc2)n1 ; Nc1cc(=O)[nH]c(SCC(=O)Nc2ccc(O)cc2)n1] 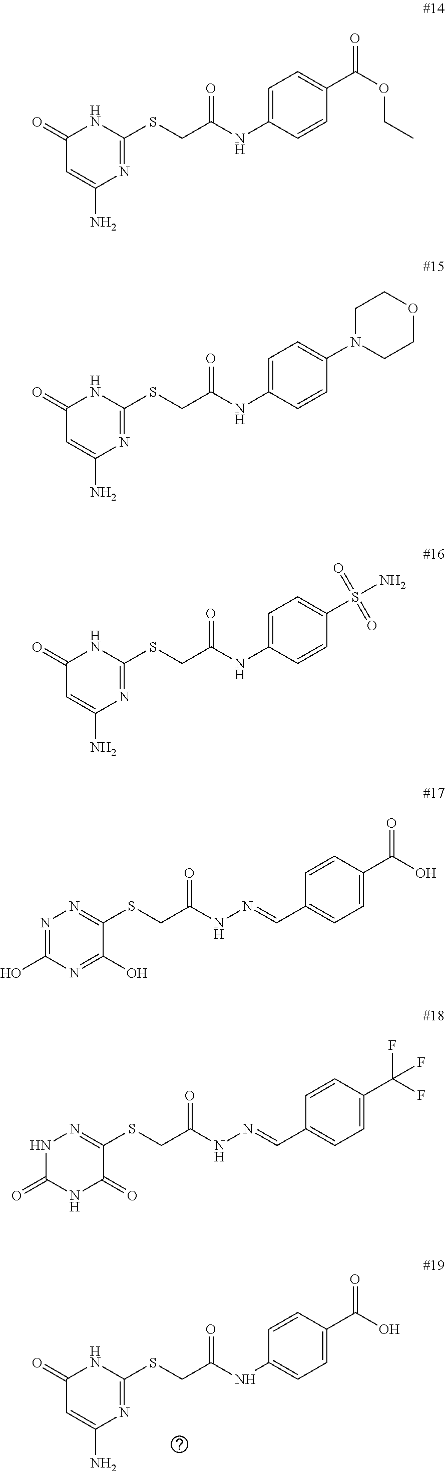 [SMILES: CCOC(=O)c1ccc(NC(=O)CSc2nc(N)cc(=O)[nH]2)cc1 ; Nc1cc(=O)[nH]c(SCC(=O)Nc2ccc(N3CCOCC3)cc2)n1 ; Nc1cc(=O)[nH]c(SCC(=O)Nc2ccc(S(N)(=O)=O)cc2)n1 ; O=C(CSc1nnc(O)nc1O)N/N=C/c1ccc(C(=O)O)cc1 ; O=C(CSc1n[nH]c(=O)[nH]c1=O)N/N=C/c1ccc(C(F)(F)F)cc1 ; Nc1cc(=O)[nH]c(SCC(=O)Nc2ccc(C(=O)O)cc2)n1] 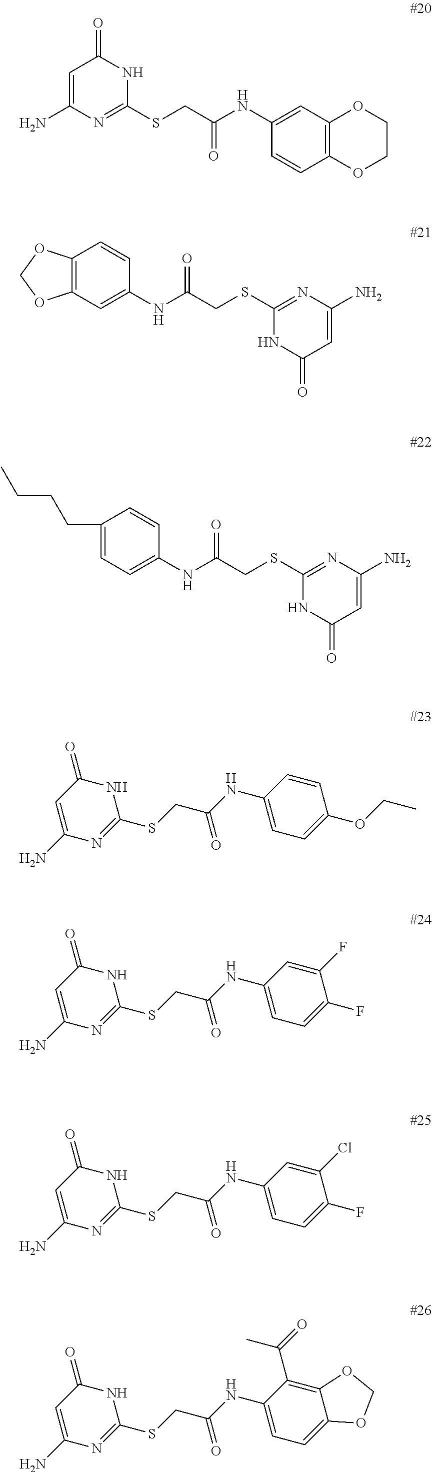 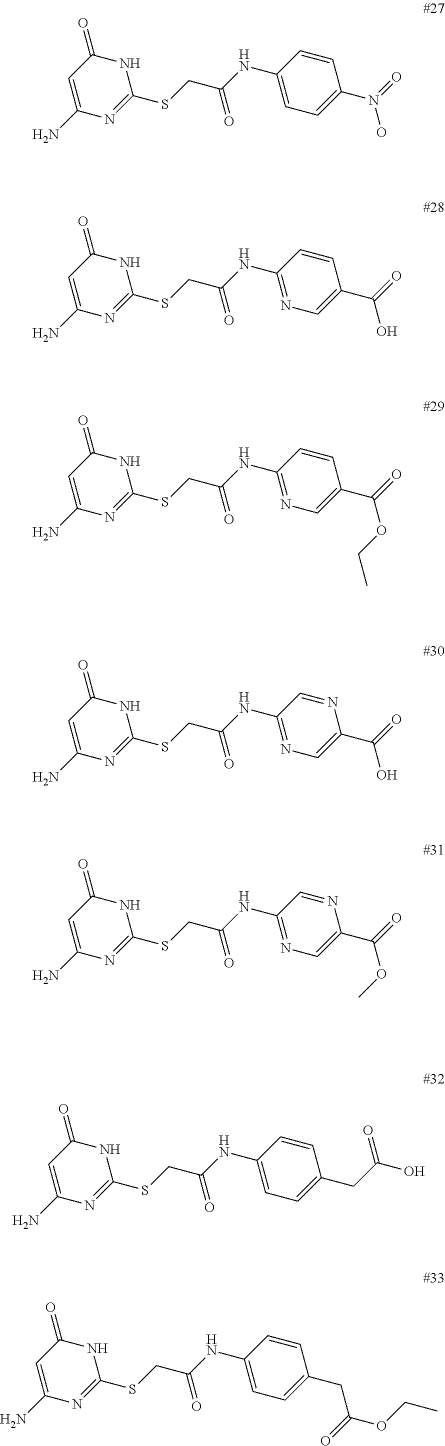 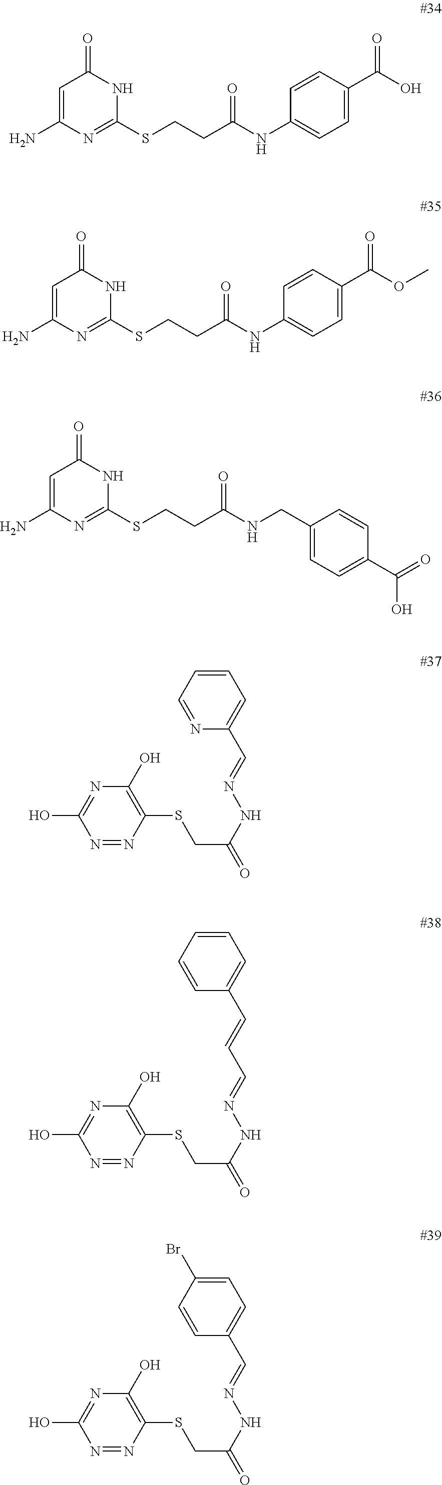 [SMILES: Nc1cc(=O)[nH]c(SCCC(=O)Nc2ccc(C(=O)O)cc2)n1 ; COC(=O)c1ccc(NC(=O)CCSc2nc(N)cc(=O)[nH]2)cc1 ; Nc1cc(=O)[nH]c(SCCC(=O)NCc2ccc(C(=O)O)cc2)n1 ; O=C(CSc1nnc(O)nc1O)N/N=C/c1ccccn1 ; O=C(CSc1nnc(O)nc1O)N/N=C/C=C/c1ccccc1 ; O=C(CSc1nnc(O)nc1O)N/N=C/c1ccc(Br)cc1] 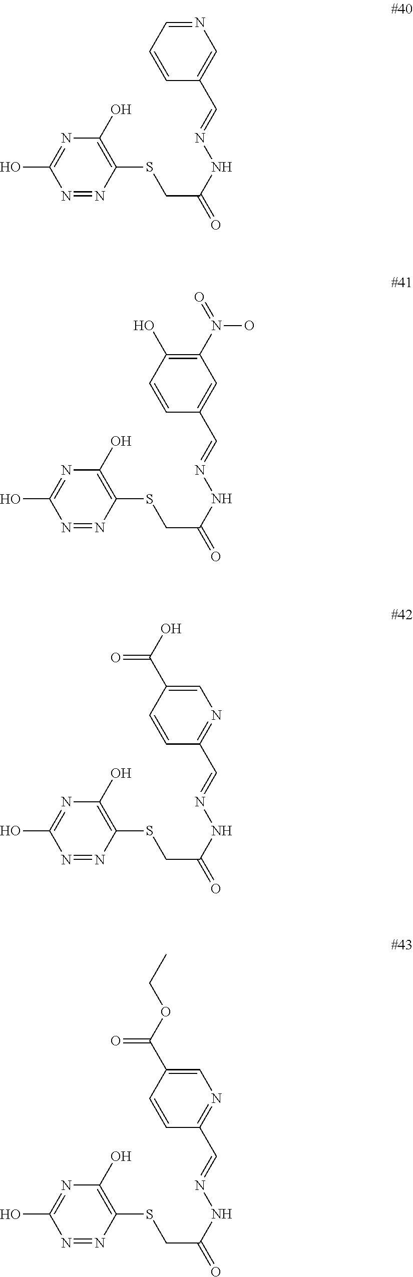 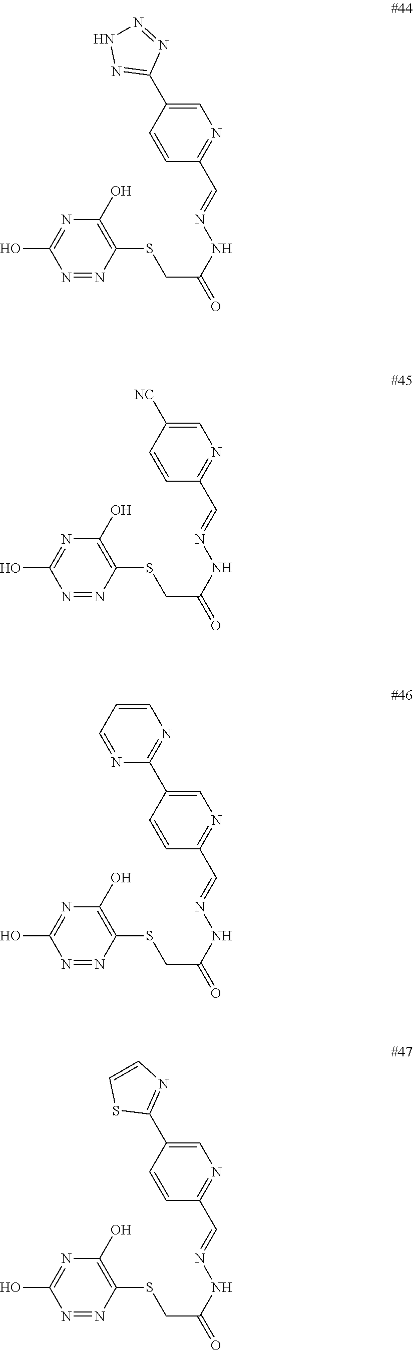 [SMILES: O=C(CSc1nnc(O)nc1O)N/N=C/c1ccc(-c2nn[nH]n2)cn1 ; N#Cc1ccc(/C=N/NC(=O)CSc2nnc(O)nc2O)nc1 ; O=C(CSc1nnc(O)nc1O)N/N=C/c1ccc(-c2ncccn2)cn1 ; O=C(CSc1nnc(O)nc1O)N/N=C/c1ccc(-c2nccs2)cn1] 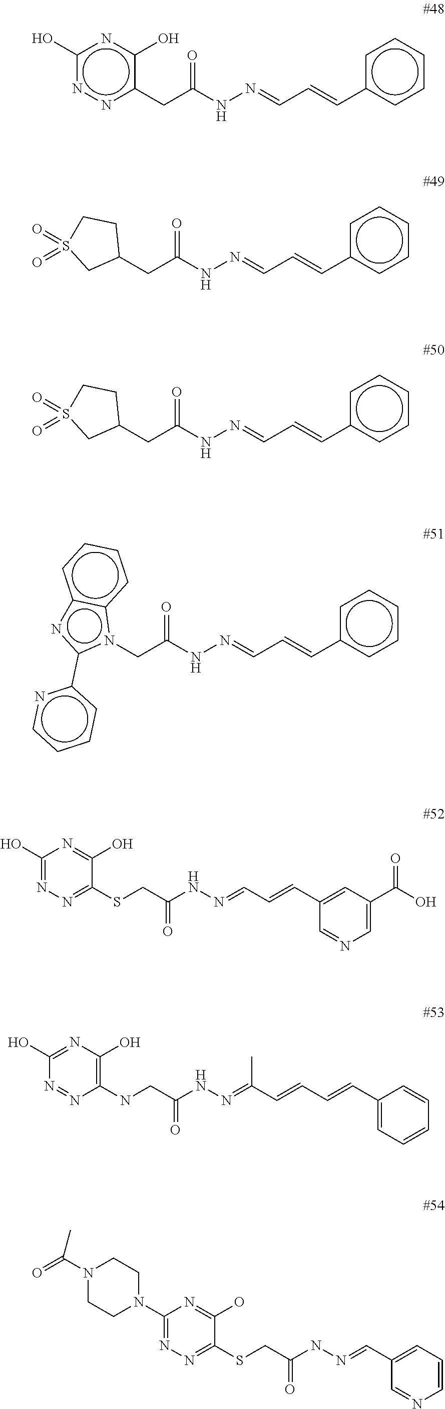 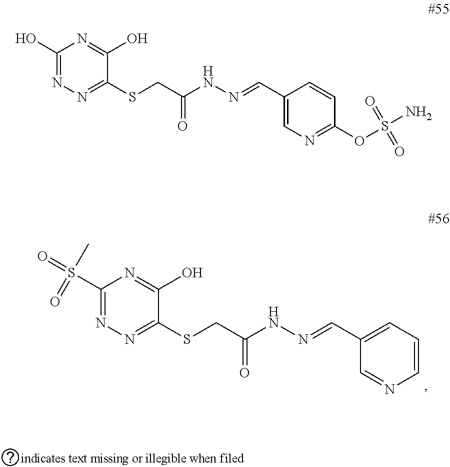 [SMILES: NS(=O)(=O)Oc1ccc(/C=N/NC(=O)CSc2nnc(O)nc2O)cn1 ; CS(=O)(=O)c1nnc(SCC(=O)N/N=C/c2cccnc2)c(O)n1]
salts thereof, and functional derivatives and thereof.

13. (canceled)

14. A pharmaceutical composition comprising the compound of claim 1 and a pharmaceutically acceptable carrier.

15. A method of treating, inhibiting, or preventing HIV infection comprising administering a therapeutically effective amount of a compound of formula (I): 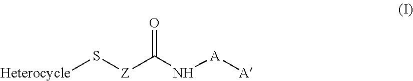
or a pharmaceutically acceptable salt thereof to a subject, wherein:
A is absent or is —N═CH—;
Z is —CH₂— or —CH₂—CH₂—;
A′ is C₆-C₁₀ aryl or C₆-C₁₀ heteroaryl, optionally substituted with one or more of C₁-C₆ alkyl, —OH, —O—C₁-C₆ alkyl, —OCX₃, halogen, —C(O)OR, —SO₂NR′R″, —NR′R″ or —O—(CH₂)₁₋₃—O— to form a bicyclic ring; wherein X is halogen, R is C₁-C₆ alkyl, R′ and R″ are independently H, C₁-C₆ alkyl, or R′ and R″ taken together with N form a C₃-C₇ heterocycle comprising one or more additional hetero atoms selected from N and O; and
heterocycle(s) are substituted or unsubstituted;
wherein the compound comprises a pharmacophore functionally corresponding with pharmacophore-I of FIG. 2.

16. The method of claim 15, wherein the compound of formula (I) is selected from the group consisting of compound #1, #2, #3, #4, #5, #6, #7, #8, #9, #10, #11, #12, #13, #14, #15, #16, #17, #18, #19, #20, #21, #22, #23, #24, #25, #26, #27, #28, #29, #30, #31, #32, #33, #34, #35, #36, #37, #38, #39, #40, #41, #42, #43, #44, #45, #46, #47, #48, #49, #50, #51, #52, #53, #54, #55, #56, salts thereof, and functional derivatives thereof.

17. (canceled)

18. The method of claim 16, wherein treating HIV infection comprises HIV entry inhibition.

19. (canceled)

20. (canceled)

21. (canceled)

22. A method of identifying a compound for binding with the glycosphingolipids (GSL) binding site at the V3-loop tip of gp120 of HIV, and for inducing the conformational change following chemokine receptor binding to inhibit chemokine receptor binding, said method comprising:
screening a small molecule library using in silico high throughput docking for candidate compounds having a conformation capable of targeting and disrupting co-receptor binding or having a pharmacophore functionally corresponding with pharmacophore I of FIG. 2; and
evaluating the candidate compounds for the ability to inhibit HIV infection to identify a compound for binding with the GSL binding site.

23. The method of claim 22 wherein the small molecule library is screened for candidate compounds having a pharmacophore functionally corresponding with pharmacophore I of FIG. 2.

24. The method of claim 22, wherein the pharmacophore comprises a negatively ionizable feature, a hydrophobic aromatic feature, a hydrogen bond acceptor feature, and a hydrogen bond donor feature.

25. The method of claim 24, wherein the pharmacophore comprises inter-feature distances of:
between the negatively ionizable feature and the hydrophobic aromatic feature a distance of from about 3 to about 5 Å, or from about 3.5 to about 4.5 Å;
between the hydrophobic aromatic feature and the hydrogen bond acceptor feature a distance of from about 7 to about 11 Å, or from about 8 to about 10 Å;
between the negatively ionizable feature and the hydrogen bond donor feature a distance of from about 7 to about 11 Å, or from about 8 to about 10 Å; and
between the hydrogen bond donor feature and the hydrogen bond acceptor feature a distance of from about 4 to about 8 Å, or from about 5 to about 7 Å.

26. The method of claim 22, wherein the pharmacophore comprises pharmacophore-1A:
(NI-AR)-EV-(HDₙ-HAₘ) (pharmacophore-1A)
wherein:
NI is a negatively ionizable feature;
AR is a hydrophobic aromatic ring feature;
EV is an excluded volume of at least 3 Å;
HD is a hydrogen bond donor feature, wherein n is 1 or 2;
HA is a hydrogen bond acceptor feature, wherein m is 1 or 2;
the inter-feature distance between NI and AR is from about 3.5 to 4.5 Å; and
the inter-feature distance between HD and HA is from about 5 to about 7 Å.
